(19) United States
(12) Patent Application Publication
(10) Pub. No.: US 2024/0016609 A1
(43) Pub. Date: Jan. 18, 2024

(54) MITRAL VALVE IMPLANTS

(71) Applicant: SHIFAMED HOLDINGS, LLC, Campbell, CA (US)

(72) Inventors: Jasper Ellington ADAMEK-BOWERS, Campbell, CA (US); Cornelius CROWLEY, San Francisco, CA (US); Amr SALAHIEH, Saratoga, CA (US); Troy THORNTON, San Francisco, CA (US); Philip PARK, Campbell, CA (US)

(21) Appl. No.: 18/255,763

(22) PCT Filed: Dec. 3, 2021
(86) PCT No.: PCT/US2021/061787

§ 371 Date: Jun. 2, 2023

Related U.S. Application Data
(60) Provisional application No. 63/120,854, filed on Dec. 3, 2020.
(60) Provisional application No. 63/158,831, filed on Mar. 9, 2021.

(51) Int. Cl. A61F 2/24

(52) U.S. Cl. CPC A61F 2/2487; A61F 2/2418; A61F 2/2445; A61F 2250/0063; A61F 2220/0016; A61F 2220/0075

(57) ABSTRACT

Implants and methods for treating diseased heart valves. The implants may include a support member configured for placement near the diseased valve and an anchoring system coupled to the support member. The support member may be used to treat regurgitation and/or support a valve replacement therein. The support member may have an annular shape with an opening configured to generally align with an opening of the diseased valve. In some examples, the support member includes a coaptation structure configured to seal with the native leaflets. The anchoring system may include one or more tethers anchored to tissue of a ventricle, such as a ventricle wall and/or a papillary muscle. In some examples, sufficient tension is applied on the tethers to stabilize or reduce ventricle dilation.

### CROSS REFERENCE TO RELATED APPLICATIONS

This application claims priority to U.S. Provisional Application No. 63/158,831, entitled “MITRAL VALVE IMPLANTS,” filed on Mar. 9, 2021, and to U.S. Provisional Patent Application No. 63/120,854, entitled “TETHERED STRUCTURAL HEART REMODELING,” filed on Dec. 3, 2020, each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Heart failure (HF) is a medical condition associated with the inability of the heart to effectively pump blood to the body. Heart failure affects millions of people worldwide, and may arise from multiple root causes, but is generally associated with myocardial stiffening, myocardial shape remodeling, and/or abnormal cardiovascular dynamics. Shape remodeling may include an increase in size in a heart ventricle, for example, ventricular dilation. Patients with valvular disease have abnormal anatomy and/or function of at least one valve. For example, a valve may suffer from insufficiency, also referred to as regurgitation, when the valve does not fully close and allows blood to flow retrograde. Valve stenosis can cause a valve to fail to open properly. Other diseases may also lead to dysfunction of the valves. Leaflets of the valve may fail to properly coapt (seal against one another) due to altered anatomy of the annulus, leaflets, and/or ventricle. While medications may be used to treat the disease, in many cases the defective valve may need to be repaired or replaced at some point during the patient's lifetime.

### SUMMARY OF THE DISCLOSURE

Described herein are systems, devices, and methods for treating a diseased valve (e.g., mitral valve). In some cases, the systems, devices, and methods are used to treat regurgitation and/or for performing valve replacement. In some cases, the system is used to repair aspects of a valve. In some cases, the system is used to replace a valve, or portions of the valve. In some embodiments, the systems described herein are delivered in a minimally invasive manner, for example endovascularly via one or more delivery catheters. In some embodiments, the systems described herein may be delivered via a transseptal approach.

In one aspect, it may be beneficial to provide a support member and anchored tether system to at least partially remodel a heart ventricle (e.g., reduce a size thereof), to remodel a portion of a native valve (e.g., annulus), and/or to provide a support structure for implantation of a prosthetic valve to improve heart function. In one aspect, it may be beneficial to provide a support member, a mating body, and anchored tether system to improve coaptation of native valve leaflets to improve heart function. In one aspect, it may be beneficial to provide an expandable frame in an atrium of a heart, and a support member operatively coupled with the frame and placed near a native valve annulus to improve heart function.

One aspect of the disclosure is a system for treating a diseased heart, the system comprising: an annular support member that is shaped and sized for placement near a native valve annulus; an anchor member comprising at least one anchor portion configured for implantation in tissue within a ventricle; and at least one tether that is configured for extension from the at least one anchor portion to the support member, the at least one tether configured to apply a tensioning force between the support member and the at least one anchor portion.

In this aspect, the at least one anchor portion may be configured for implantation in a ventricle wall or a papillary muscle.

In this aspect, the support member may be configured to alter a dimension of the native valve annulus.

In this aspect, the at least one tether may be configured to secure the support member to the native valve annulus.

In this aspect, the at least one anchor portion may comprise a coil near a distal end thereof.

In this aspect, the at least one anchor portion may comprise a taper that reduces to a point at a distal end thereof.

In this aspect, the at least one anchor portion may include an elongate tube having a first circumference, and wherein the at least one anchor portion comprises an expandable region configured to expand to a second circumference that is larger than the first circumference, wherein the expandable region is near a distal end of the at least one anchor portion.

In this aspect, the at least one anchor portion may comprise at least two expandable regions configured to expand to circumferences larger than the first circumference.

In this aspect, the support member may comprise a self-expanding material.

In this aspect, the system may further comprise a prosthetic valve that is sized and shaped to be placed within the support member.

In this aspect, the prosthetic valve may be configured to transition from an unexpanded configuration to an expanded configuration.

In this aspect, upon placement within the support member, the prosthetic valve may be configured to displace native valve leaflets.

In this aspect, the system may further comprise first and second flow meters that are coupled with the support member, and that are positioned and configured for inductive coupling.

In this aspect, the first and second flow meters may be disposed about a periphery of the support member.

In this aspect, the annular support member may be expandable and includes wires arranged to form an annular shape.

In this aspect, the wires may define an inner wall and outer wall.

In this aspect, the annular support member may be conically shaped.

In this aspect, the annular support member may be half-dome shaped.

In this aspect, the at least one anchor may include an expandable portion that is configured to expand from a first diameter to a second diameter.

In this aspect, the at least one anchor may include a collapsible tube having a pattern of cutouts that is configured to collapse the tube and form radially extending arms.

One aspect of the disclosure is a method of securing a support member near a native valve annulus of a heart, comprising: securing at least one anchor portion of an anchor member to tissue of a ventricle, the anchor member comprising a tether portion extending at least partially through the native valve annulus; delivering an annular support member to a position near the native valve annulus; coupling the support member with the tether portion; and applying a tension on the tether portion to provide a tensioning force between the at least one anchor portion and the support member.

In this aspect, delivering the support member may comprise securing the support member to the valve annulus and/or to a portion of the wall of the heart adjacent the valve annulus.

In this aspect, delivering the support member may comprise positioning the support member in a supra-annular position.

In this aspect, applying tension on the tether portion may comprise changing a dimension of the ventricle and/or the valve annulus.

In this aspect, applying tension on the tether portion may apply a pulling force on a wall of the ventricle to reduce a volume of the ventricle.

In this aspect, applying tension on the tether portion may cause a reduction in a size of the valve annulus.

In this aspect, the coupling the support member with the tether portion may be prior to delivering the support member.

In this aspect, delivering the support member may be to a left atrium of the heart.

In this aspect, the method may further comprise delivering a prosthetic valve to the support member.

In this aspect, delivering the prosthetic valve may comprise expanding the prosthetic valve from an unexpanded configuration to an expanded configuration to displace native leaflets of the valve.

In this aspect, the tether portion may extend through a commissure of the native valve.

In this aspect, securing the at least one anchor portion may comprise securing the at least one anchor portion to one or more of a ventricle wall and a papillary muscle.

In this aspect, securing the at least one anchor portion may comprise expanding an expandable portion of the at least one anchor portion.

In this aspect, expanding the expandable portion may comprise radially extending arms of a collapsible tube.

One aspect of the disclosure is a system for treating a diseased heart, the system comprising: an annular support member that is shaped and sized for placement near a native valve annulus, the support member having an opening; a mating body coupled to the support member and disposed within the opening such that a length of the mating body is positioned between native leaflets, the mating body arranged to provide a seal against the native leaflets; and an anchor member comprising at least one anchor portion configured for implantation in a ventricle wall, and at least one tether configured to extend from the at least one anchor portion to the support member.

In this aspect, the mating body may comprise a compliant material that is configured to accommodate a shape of the native leaflets.

In this aspect, the compliant material may comprise one or more of a fluid filled polymer, ePTFE and covered nitinol.

In this aspect the at least one tether may be configured to reduce a volume of the ventricle.

In this aspect, the at least one anchor may include an expandable portion that is configured to expand from a first diameter to a second diameter.

In this aspect, the at least one anchor may include a collapsible tube having a pattern of cutouts that is configured to collapse the tube and form radially extending arms.

One aspect of the disclosure is a system for treating a diseased valve, the system comprising: an annular support configured for placement adjacent to the diseased valve; an anchoring device configured for securing to an internal ventricle wall; and one or more tethers configured to tether the annular support to the anchoring device, the one or more tethers configured to provide a tensioning force on the annular support implanted adjacent to the diseased valve toward the anchoring device secured to the ventricle wall.

In this aspect, the tensioning force may be configured to stabilize and reduce ventricle dilation.

In this aspect, the tensioning force may be configured to reduce an annular dimension of the diseased valve.

In this aspect, the anchoring device may include an expandable portion that is configured to expand from a first diameter to a second diameter.

In this aspect, the anchoring device may include a collapsible tube having a pattern of cutouts that is configured to collapse the tube and form radially extending arms.

One aspect of the disclosure is an implant for treating a diseased valve, the implant comprising: a frame configured for placement adjacent to the diseased valve and having a central opening; a coaptation structure coupled to the frame and extending radially within the opening of the frame, the coaptation structure arranged for placement within the diseased valve for coaptation with native leaflets of the diseased valve; and an anchoring tab extending from the frame, the anchoring tab configured to secure the implant to the diseased valve.

In this aspect, the anchoring tab may include a hook portion configured to wrap around the native leaflets, pass through the native leaflets, or wrap around and pass through the native leaflets.

In this aspect, the hook portion may extend from a ventricle side of the implant.

In this aspect, the hook portion may be configured to couple with a tether coupled to an anchor secured to the ventricle wall.

In this aspect, the anchoring tab may extend within the central opening of the frame and radially inward with respect to the frame.

In this aspect, the anchoring tab may be arranged for placement within or near a commissure of the native leaflets.

In this aspect, the implant may include a first anchoring tab arranged for placement within or near the anterior commissure of the native leaflets, and a second anchoring tab arranged for placement within or near the posterior commissure of the native leaflets.

In this aspect, the frame may include a lip around a perimeter of the frame that is sized and shaped to promote ingrowth of tissue thereon.

In this aspect, the implant may further comprise a tether and an anchor, the having a proximal end coupled to the anchoring tab and a distal end coupled to the anchor, wherein the anchor is secured to tissue of a ventricle of the heart.

In this aspect, the anchor may include an expandable portion that is configured to expand from a first diameter to a second diameter.

In this aspect, the anchor may include a collapsible tube having a pattern of cutouts that is configured to collapse the tube and form radially extending arms.

One aspect of the disclosure is an implant for treating a diseased valve, the implant comprising: a frame comprising an opening having an inner wall and configured for placement adjacent to the diseased valve, wherein the frame is configured to transition to a radially expanded configuration upon application of a radially outward pressure to the inner wall, and to contract to a radially reduced configuration upon release of the radially outward pressure; and a series of hooks coupled with the frame and configured to secure the implant to the diseased valve, the series of hooks extending from a first side of the frame and arranged radially around the central opening of the frame, a least a portion of the series of hooks curved radially inward and configured to engage with tissue upon expansion of the frame and remain engaged with the tissue upon contraction of the frame.

In this aspect, a central opening of the frame may have a diameter that is less than a dimeter of an opening of the diseased valve.

In this aspect, the diameter of the central opening may range from 5% to 95% of the diameter of the diseased valve.

In this aspect, the frame may be made of a shape-memory material.

In this aspect, the frame may be made of a shape-memory wire.

In this aspect, the implant may further comprise a series of outward radiating petals configured to provide structural stiffness to the frame.

In this aspect, at least a portion of the implant may include a covering configured to promote ingrowth of tissue onto the implant.

In this aspect, the first side of the frame may be a ventricle side of the frame.

In this aspect, each of the series of hooks may be curved radially inward and is configured to engage with tissue upon expansion of the frame and remain engaged with the tissue upon contraction of the frame.

In this aspect, the frame may be in a lower energy state when in the radially reduced configuration compared to when the frame is in the radially expanded configuration.

In this aspect, the frame may be configured to compress into a delivery configuration within a delivery catheter.

In this aspect, the implant may further comprise a tether and an anchor, the having a proximal end coupled to the implant and a distal end coupled to the anchor, wherein the anchor is secured to tissue of a ventricle of the heart.

In this aspect, the anchor may include an expandable portion that is configured to expand from a first diameter to a second diameter.

In this aspect, the anchor may include a collapsible tube having a pattern of cutouts that is configured to collapse the tube and form radially extending arms.

One aspect of the disclosure is a method of treating a diseased valve, comprising: positioning an implant within the diseased valve using a delivery catheter with the implant positioned therein, the implant comprising a frame with an inner wall defining an opening, and a series of hooks coupled to the frame and arranged radially around the opening; radially expanding the frame by applying a radially outward pressure to the inner wall, wherein expanding the frame causes at least a portion of the series of hooks to engage with tissue of the diseased valve or around the diseased valve; and releasing the radially outward pressure from the inner wall to cause the frame to contract to a radially reduced deployed configuration, wherein the at least a portion of the series of hooks remain engaged with the tissue upon contraction of the frame, thereby securing the implant to the diseased valve.

In this aspect, applying the radially outward pressure to the inner wall may comprise inflating a balloon within the opening of the frame such that the balloon applies pressure against the inner wall of the frame.

In this aspect, releasing the radially outward pressure may comprise deflating the balloon.

In this aspect, method may further comprise allowing blood to flow through the opening of the frame within the diseased valve as the balloon is inflated and deflated.

In this aspect, method may further comprise removing the balloon from the opening of the frame.

In this aspect, the method may further comprise inserting a prosthetic valve within the opening of the frame.

In this aspect, the series of hooks may be curved radially inward such that the hooks remain engaged with the tissue upon contraction of the frame.

In this aspect, the method may further comprise connecting the implant via a tether to an anchor secured to tissue within a ventricle of the heart.

In this aspect, the anchor may include an expandable portion that is configured to expand from a first diameter to a second diameter.

In this aspect, the anchor may include a collapsible tube having a pattern of cutouts that is configured to collapse the tube and form radially extending arms.

One aspect of the disclosure is a system for treating a diseased heart, the system comprising: a support member (e.g., dock) comprising a body that is shaped and sized for placement near a native valve annulus, and accommodation of native valve leaflet function; and an anchor member comprising at least one anchor portion configured for implantation in a ventricle wall, and an elongate tether portion that is configured for extension from the at least one anchor portion to the support member, and to couple therewith; wherein the elongate tether portion in a first configuration is configured to accommodate relative movement of the support member therewith, and in a second configuration is configured to secure the support member near the native valve annulus.

In this aspect, the elongate tether portion in the second configuration may be configured to urge the support member to alter a dimension of the native valve annulus.

In this aspect, the elongate tether portion in the second configuration may be configured to urge the anchor member to alter a dimension of the ventricle wall.

In this aspect, the elongate tether portion in the second configuration may be further configured to secure the support member to the native valve annulus.

In this aspect, the at least one anchor portion may comprise a coil near a distal end thereof.

In this aspect, the at least one anchor portion may comprise a taper that reduces to a point at a distal end thereof.

In this aspect, the at least one anchor portion may be generally an elongate tube having a first circumference, and wherein the at least one anchor portion comprises a region having a second circumference that is larger than the first circumference, near a distal end thereof.

In this aspect, the at least one anchor portion may comprise at least two regions having circumferences larger than the first circumference.

In this aspect, the support member may comprise a self-expanding material (e.g., nitinol).

In this aspect, the system may further comprise a prosthetic valve that is sized and shaped to be placed within the support member.

In this aspect, the prosthetic valve may comprise an unexpanded configuration for delivery thereof, and an expanded configuration for placement within the support member.

In this aspect, upon placement within the support member, the prosthetic valve may be configured to displace native valve leaflets.

In this aspect, the system may further comprise first and second flow meters that are coupled with the support member, and that are positioned and configured for inductive coupling.

In this aspect, the first and second flow meters may be disposed about a periphery of the support member.

One aspect of the disclosure is a method of securing a support member near a native valve annulus of a heart, comprising: securing at least one anchor portion of an anchor member to a wall of the ventricle, the anchor member comprising a tether portion extending at least partially through the valve annulus; delivering a support member (e.g., dock or ring) to a position near the valve annulus; coupling the support member with the tether portion; tensioning the tether portion to urge the at least one anchor portion and the support member toward each other; and securing the support member near the native valve annulus.

In this aspect, securing the support member may comprise securing to the valve annulus and/or to a portion of the wall of the heart adjacent the valve annulus.

In this aspect, securing the support member may be to a supra-annular position.

In this aspect, tensioning the tether portion may comprise changing a dimension of a ventricle and/or a valve annulus of a heart.

In this aspect, tensioning may comprise pulling the wall of the ventricle to reduce the dimension thereof.

In this aspect, tensioning the tether portion may further comprise reducing a dimension of the valve annulus.

In this aspect, the coupling the support member with the tether portion may be prior to delivering the support member.

In this aspect, delivering the support member may be to a left atrium of the heart.

In this aspect, the method may further comprise delivering a prosthetic valve to the support member.

In this aspect, delivering the prosthetic valve may comprise expanding from an unexpanded configuration to an expanded configuration to displace native leaflets of the valve.

In this aspect, the tether portion may extend through a commissure of the valve.

One aspect of the disclosure is a system for treating a diseased heart, the system comprising: a support member (e.g., dock) comprising a body that is shaped and sized for placement near a native valve annulus, and having an opening for accommodation of native valve leaflet function; a mating body (also referred to as a coaptation structure or blocker) coupled to the support member and disposed within the opening such that a length of the mating body extends generally between native leaflets, the mating body configured for providing a seal for the native leaflets (e.g., coaptation therewith); and an anchor member comprising at least one anchor portion configured for implantation in a ventricle wall, and an elongate tether portion that is configured for extension from the at least one anchor portion to the support member, and to couple therewith; wherein the elongate tether portion in a first configuration is configured to accommodate relative movement of the support member therewith, and in a second configuration is configured to secure the support member near the native valve annulus.

In this aspect, the mating body may comprise a compliant material (e.g., fluid-filled polymer, ePTFE-covered nitinol) that is configured to accommodate a shape of the native leaflets.

In this aspect, the elongate tether portion in the second configuration may further be configured to reduce a dimension of the ventricle wall.

One aspect of the disclosure is a system for treating a diseased heart, the system comprising: a frame comprising a body that is configured to transition between at least a first configuration in which the frame has a reduced delivery profile, and a second expanded configuration in which the frame is shaped and sized for placement within an atrium of the heart, and for at least partial support by 1) a wall thereof and/or 2) a surface of a native valve annulus that is in fluid communication with the atrium; and a support (e.g., dock) configured for operative coupling with the frame and comprising a body that is shaped and sized for placement near the native valve annulus, the support having an opening for accommodation of native valve leaflet function; wherein when the system is implanted within the atrium of the heart, the support is positioned near the native valve annulus.

In this aspect, the frame may be self-expandable from the first configuration to the second expanded configuration.

In this aspect, in the second expanded configuration, the frame may be configured to anchor the support near the native valve annulus.

In this aspect, the support may further comprise a valve support that is configured to retain a prosthetic valve.

In this aspect, the method may further comprise a prosthetic valve supported within the valve support.

One aspect of the disclosure is a system for treating a diseased valve, the system comprising: a valve implant configured for placement adjacent to the diseased valve; an anchoring device configured for securing to an internal ventricle wall; and one or more tethers configured to tether the valve implant to the anchoring device, the one or more tethers configured to provide a tensioning force on the valve implant implanted adjacent to the diseased valve toward the anchoring device secured to the ventricle wall.

In this aspect, the tensioning force may be configured to stabilize and reduce ventricle dilation.

In this aspect, the tensioning force may be configured to reduce an annular dimension of the diseased valve.

One aspect of the disclosure is an implant for treating a diseased valve, the implant comprising: a frame configured for placement adjacent to the diseased valve and having a central opening; a coaptation structure coupled to the frame and extending radially within the opening of the frame, the coaptation structure arranged for placement within the diseased valve for coaptation with native leaflets of the diseased valve; and an anchoring tab extending from the frame, the anchoring tab configured to secure the implant to the diseased valve.

In this aspect, the anchoring tab may include a hook portion configured to wrap around the native leaflets, pass through the native leaflets, or wrap around and pass through the native leaflets.

In this aspect, the hook portion may extend from a ventricle side of the implant.

In this aspect, the hook portion may be configured to couple with a tether coupled to an anchor secured to the ventricle wall.

In this aspect, the anchoring tab may extend within the central opening of the frame and radially inward with respect to the frame.

In this aspect, the anchoring tab may be arranged for placement within or near a commissure of the native leaflets.

In this aspect, the implant may include a first anchoring tab arranged for placement within or near the anterior commissure of the native leaflets, and a second anchoring tab arranged for placement within or near the posterior commissure of the native leaflets.

In this aspect, the frame may include a lip around a perimeter of the frame that is sized and shaped to promote ingrowth of tissue thereon.

One aspect of the disclosure is an implant for treating a diseased valve, the implant comprising: a frame comprising an opening having an inner wall and configured for placement adjacent to the diseased valve, wherein the frame is configured to transition to a radially expanded configuration upon application of a radially outward pressure to the inner wall, and to contract to a radially reduced configuration upon release of the radially outward pressure; and a series of hooks coupled with the frame and configured to secure the implant to the diseased valve, the series of hooks extending from a first side of the frame and arranged radially around the central opening of the frame, a least a portion of the series of hooks curved radially inward and configured to engage with tissue upon expansion of the frame and remain engaged with the tissue upon contraction of the frame.

In this aspect, a central opening of the frame may have a diameter that is less than a dimeter of an opening of the diseased valve.

In this aspect, the diameter of the central opening may range from 5% to 95% of the diameter of the diseased valve.

In this aspect, the frame may be made of a shape-memory material.

In this aspect, the frame may be made of a shape-memory wire.

In this aspect, the implant may further comprise a series of outward radiating petals configured to provide structural stiffness to the frame.

In this aspect, at least a portion of the implant may include a covering configured to promote ingrowth of tissue onto the implant.

In this aspect, the first side of the frame may be a ventricle side of the frame.

In this aspect, each of the series of hooks may be curved radially inward and is configured to engage with tissue upon expansion of the frame and remain engaged with the tissue upon contraction of the frame.

In this aspect, the frame may be in a lower energy state when in the radially reduced configuration compared to when the frame is in the radially expanded configuration.

In this aspect, the frame may be configured to compress into a delivery configuration within a delivery catheter.

One aspect of the disclosure is a method of treating a diseased valve, comprising: positioning an implant within the diseased valve using a delivery catheter with the implant positioned therein, the implant comprising a frame with an inner wall defining an opening, and a series of hooks coupled to the frame and arranged radially around the opening; radially expanding the frame by applying a radially outward pressure to the inner wall, wherein expanding the frame causes at least a portion of the series of hooks to engage with tissue of the diseased valve or around the diseased valve; and releasing the radially outward pressure from the inner wall to cause the frame to contract to a radially reduced deployed configuration, wherein the at least a portion of the series of hooks remain engaged with the tissue upon contraction of the frame, thereby securing the implant to the diseased valve.

In this aspect, applying the radially outward pressure to the inner wall may comprise inflating a balloon within the opening of the frame such that the balloon applies pressure against the inner wall of the frame.

In this aspect, releasing the radially outward pressure may comprise deflating the balloon.

In this aspect, the method may further comprise allowing blood to flow through the opening of the frame within the diseased valve as the balloon is inflated and deflated.

In this aspect, the method may further comprise removing the balloon from the opening of the frame.

In this aspect, the method may further comprise inserting a prosthetic valve within the opening of the frame.

In this aspect, the series of hooks may be curved radially inward such that the hooks remain engaged with the tissue upon contraction of the frame.

One aspect of the disclosure is an anchoring device for securing to a heart wall, the anchoring device comprising: a central support including an opening; and a compression coil having a first annular coiled portion connected to a second annular coiled portion via a straight central connection portion, wherein the straight central connection portion of the compression coil is positioned through the opening of central support such that the first annular coiled portion is on a first side of the central support, and the second annular coiled portion is on a second side of the central support, wherein when the anchoring device is in a deployed configuration: the compression coil is configured to pass through the heart wall such that first coiled portion is positioned within the heart and the second coiled portion is positioned outside of the heart; and the first and second coiled portions are configured to apply a clamping force against the heart wall to secure the anchoring device to the heart wall.

In this aspect, the anchoring device may further comprise an annular washer engaged with and axially aligned with the central support, the annular washer radially extending outward from the central support, wherein when the anchoring device is in a deployed configuration, the washer is configured to radially distribute the clamping force applied to the heart wall.

In this aspect, the annular washer may include a convex surface configured to contact the heart wall.

In this aspect, the anchoring device may be configured to transition from a delivery configuration to the deployed configuration, wherein when the anchor is in a delivery configuration, the compression coil has a straightened shape with a sufficiently small profile for residing within a delivery catheter.

In this aspect, the compression coil may be made of a shape-memory material.

In this aspect, the anchoring device may be configured to couple with one or more tethers that are coupled to a valve implant, wherein the one or more tethers are configured to provide a tensioning force between the valve implant and the anchoring device.

One aspect of the disclosure is a method of securing an anchoring device to a heart wall, the method comprising: advancing a delivery catheter within the heart toward an anchoring site of the heart wall, wherein an inner lumen of the delivery catheter includes a central support of the anchoring device positioned therein; positioning the central support with respect to an anchoring site along the inner surface of the heart wall, wherein an opening of the central support is aligned with a target puncture location; advancing a wire of the anchoring device through the delivery catheter and the opening of the central support such that the wire passes through the heart wall at the target puncture location, wherein a distal portion of the wire passes through the heart wall and takes on a first coiled annular shape outside of the heart wall; and retracting the delivery catheter from the anchoring site such that a proximal portion of the wire takes on a second coiled annular shape inside the heart wall, wherein, when the wire is fully deployed from the delivery catheter, the distal and proximal portions of the wire cooperate to apply a clamping force against the heart wall to secure the anchoring device to the heart wall.

In this aspect, when the wire is fully deployed from the delivery catheter, a central straight portion of the wire may be positioned within the opening of the central support.

In this aspect, positioning the central support with respect to an anchoring site may include centering the central support with respect to an inner wall of the delivery catheter using one or more centering arms coupled to the central support.

In this aspect, the method may further comprise removing the one or more centering arms from the central support.

In this aspect, the method may further comprise, after passing the distal portion of the wire through the heart wall, deploying an annular washer over the anchoring site of the heart wall, the annular washer configured to radially distribute the clamping force applied to the heart wall.

In this aspect, the wherein deploying the annular washer may comprise: advancing the annular washer through the delivery catheter while in a radially contracted delivery state; and exposing the annular washer from the delivery catheter such that the annular washer expands to a radially expanded deployed state.

In this aspect, at least a portion of the annular washer may be positioned between the proximal portion of the wire and the heart wall when the wire is fully deployed.

In this aspect, passing the wire through the heart wall at the target puncture location may include puncturing the heart wall using a distal end of the wire.

In this aspect, the method may further comprise coupling one or more tethers to the anchoring device, the one or more tethers coupled to a valve implant, wherein the one or more tethers are configured to provide a tensioning force between the valve implant and the anchoring device.

One aspect of the disclosure is a system for treating a diseased valve, the system comprising: a valve implant configured for placement adjacent to the diseased valve; an anchoring device including a compression coil having a first annular portion connected to a second annular portion via a connection region, wherein the connection region of the compression coil is configured to pass through a ventricle wall such that the first annular portion is on one side of the ventricle wall and the second annular portion is on an opposing second side of the ventricle wall, wherein the first and second portions are configured to apply a clamping force against the ventricle wall to secure the anchoring device to the ventricle wall; and one or more tethers configured to tether the valve implant to the anchoring device, the one or more tethers configured to provide a tensioning force on the valve implant positioned adjacent to the diseased valve toward the anchoring device secured to the ventricle wall.

In this aspect, the anchoring device may further comprise a washer between the first annular portion of the compression coil and the ventricle wall, the washer configured to distribute the clamping force applied to the ventricle wall.

In this aspect, the connection region of the compression coil may be at a central region of the anchoring device.

In this aspect, the first and second annular portions may be configured to apply a compression force against the ventricle wall that is sufficient to reduce or prevent bleeding from the connection region of the compression coil passing through the ventricle wall.

In this aspect, the compression coil may be made of shape memory material that is biased to apply the clamping force against the ventricle wall between the first and second portions.

One aspect of the disclosure is an anchoring device for securing to a heart wall, the anchoring device comprising: an elongate body including a distal portion having one or more wings extending distally from a proximal portion of the elongate body, the one or more wings configured to be embedded within the heart wall, the one or more wings configured to transition from a delivery configuration to a deployed configuration, wherein: when the one or more wings are in the delivery configuration, the one or more wings are radially contracted and sharp distal ends of the one or more wings point in a distal direction, and when the one or more wings are in the deployed configuration, the one or more wings are radially expanded and curved such that the sharp distal ends point toward a proximal direction; and a flange coupled to the proximal portion of the elongate body, the flange configured to apply a compression force against an inner surface of the heart wall when the one or more wings are embedded within the heart wall in the deployed configuration.

In this aspect, the flange may comprise an engagement surface having one or more protruding hooks configured to engage with the inner surface of the heart wall.

In this aspect, the flange may comprise an engagement surface that having a tapered shape for engagement with a tapered inner surface of the heart.

In this aspect, the proximal portion of the elongate body may be positioned within an opening of the flange.

In this aspect, the opening of the flange and the proximal portion of the elongate body may be threaded.

In this aspect, the anchoring device further comprises a cinching nut configured to transition the one or more wings from the delivery configuration to the deployed configuration upon rotation of the cinching nut.

One aspect of the disclosure is a method of securing an anchoring device to a heart wall, the method comprising: advancing a delivery catheter within the heart toward an anchoring site of the heart wall, wherein an inner lumen of the delivery catheter includes an elongate body of the anchoring device positioned therein, the elongate body including a distal portion having one or more wings extending distally from a proximal portion of the elongate body; distally advancing the elongate body to puncture an inner surface of the heart wall using sharp distal ends of the one or more wings; radially expanding the one or more wings within the heart wall, wherein, when expanded, the one or more wings take on a curved shape such that the sharp distal ends point toward a proximal direction, thereby securing the anchoring device to the heart wall; and coupling a flange to the proximal portion of the elongate body such that the flange contacts the inner surface of the heart wall and applies a compression force against the inner surface of the heart wall.

In this aspect, coupling a flange to the proximal portion of the elongate body may comprise: advancing the flange in a contracted configuration within the delivery catheter toward the elongate body embedded within the heart wall; and releasing the flange from the delivery catheter such that the flange expands to an expanded configuration.

In this aspect, the flange may comprise an engagement surface that having a tapered shape for engagement with a tapered inner surface of the heart.

In this aspect, radially expanding the one or more wings within the heart wall may comprise rotating a cinch nut of the anchoring device.

In this aspect, method may further comprise coupling one or more tethers to the anchoring device, the one or more tethers coupled to a valve implant, wherein the one or more tethers are configured to provide a tensioning force between the valve implant and the anchoring device.

One aspect of the disclosure is a system for treating a diseased valve, the system comprising: a valve implant configured for placement adjacent to the diseased valve; an anchoring device including radially extending wing portions configured to engage with tissue within a ventricle wall, the anchoring device including a flange portion configured to apply a compression force against an inner surface of the ventricle wall to secure the anchoring device to the ventricle wall; and one or more tethers configured to tether the valve implant to the anchoring device, the one or more tethers configured to provide a tensioning force on the valve implant implanted adjacent to the diseased valve toward the anchoring device secured to the ventricle wall.

In this aspect, the flange portion may include protruding features configured to engage with tissue of the inner surface of the ventricle wall.

In this aspect, the protruding features may include a plurality of hooks.

In this aspect, the anchoring device may further comprise a cinch nut configured to cause the wing portions to expand from an elongated state to a radially expanded state upon rotation of the cinch nut.

In this aspect, the flange portion may be configured to apply the compression force sufficiently to reduce or prevent bleeding from puncture of the ventricle wall by the wings.

One aspect of the disclosure is an anchoring device for securing to a heart wall, the anchoring device comprising: an embedded member configured to pass at least partially through the heart wall; and an annular member coupled to the embedded member and configured to radially distribute a compression force applied against an inner surface of the heart wall.

In this aspect, the embedded member may be a compression coil having a first annular coiled portion connected to a second annular coiled portion via a straight central connection portion, wherein the straight central connection portion of the compression coil is positioned through an opening of central support such that the first annular coiled portion is on a first side of the central support, and the second annular coiled portion is on a second side of the central support.

In this aspect, the annular member may be an annular washer engaged with and axially aligned with the central support, the annular washer radially extending outward from the central support, wherein when the anchoring device is in a deployed configuration, the washer is configured to radially distribute the clamping force applied to the heart wall.

In this aspect, the embedded member may be an elongate body including a distal portion having one or more wings extending distally from a proximal portion of the elongate body, the one or more wings configured to be embedded within the heart wall, the one or more wings configured to transition from a delivery configuration to a deployed configuration.

In this aspect, the annular member is a flange coupled to the proximal portion of the elongate body, the flange configured to apply a compression force against an inner surface of the heart wall when the one or more wings are embedded within the heart wall in the deployed configuration.

These and other aspects are described herein.

### BRIEF DESCRIPTION OF THE DRAWINGS

Novel features of embodiments described herein are set forth with particularity in the appended claims. A better understanding of the features and advantages of the embodiments may be obtained by reference to the following detailed description that sets forth illustrative embodiments and the accompanying drawings.

FIGS. 1A and 1B illustrate section views of a heart showing an exemplary dock implant used in different use cases: FIG. 1A shows the dock implant used for mitral repair and/or left ventricle reverse remodeling; and FIG. 1B shows a replacement valve used with the dock implant.

FIGS. 2A-2D illustrate section views of a heart showing an exemplary procedure flow for anchor placement: FIG. 2A shows a first anchor being deployed within a ventricle wall; FIG. 2B shows a first tether secured to the first anchor; FIG. 2C shows a second anchor being deployed within the ventricle wall; and FIG. 2D shows a second tether secured to the second anchor.

FIGS. 3A-3D illustrate section views of a heart showing an exemplary procedure flow for dock placement based on the deployed anchors of FIGS. 2A-2D: FIG. 3A shows a dock delivery system introduced within the heart; FIG. 3B shows a dock being deployed in the left atrium; FIG. 3C shows the tethers being tensioned to reduce left ventricle volume; and FIG. 3D shows the dock and the tethers being released from the delivery system.

FIGS. 4A and 4B illustrate an exemplary dock configured for mitral valve repair and/or left ventricle reverse remodeling; FIG. 4A shows a top-down view; and FIG. 4B shows a side view.

FIGS. 5A and 5B illustrate an exemplary dock with a mitral valve implanted therein; FIG. 5A shows a top-down view; and FIG. 5B shows a perspective top view.

FIGS. 6A-6C illustrate an exemplary deployment process of an anchoring device for anchoring to the left ventricle wall; FIG. 6A shows a scaffold catheter delivering anchors to a target site along the ventricle wall; FIG. 6B shows cinching of the deployed anchors; and FIG. 6C shows release of the anchors from the scaffold catheter.

FIGS. 7A and 7B illustrate an exemplary valve implant that can act as a dock for anchors and that include a coaptation blocker: FIG. 7A shows a perspective top view of the valve implant within the heart; and FIG. 7B shows a side section view of the valve implant within the heart.

FIG. 8 illustrates a section view of a heart having an exemplary valve implant including a supra-annular cage and an annular ring.

FIG. 9 illustrates a side view of an exemplary valve implant configured for supporting sensors that send and/or receive signals from/to a transducer.

FIGS. 10A-10B illustrate an exemplary transeptal TMVR dock implant configured

for cross-ventricle anchoring within ventricle walls: FIG. 10A shows a cross section commissural view of a heart with the TMVR dock implanted therein; and FIG. 10B shows a bottom view showing the implanted dock above the native valve annulus.

FIGS. 10C and 10D illustrate cross-section views of a heart illustrating conduction effects with and without transeptal TMVR dock implant anchoring: FIG. 10C shows a heart with the TMVR dock implant anchoring; and FIG. 10D shows a hear without the TMVR dock implant anchoring.

FIGS. 11A and 11B illustrate another exemplary transeptal TMVR dock implant configured for anchoring to papillary muscles: FIG. 11A shows a cross section commissural view of a heart with the TMVR dock implanted therein; and FIG. 11B shows a bottom view showing the implanted dock above the native valve annulus.

FIGS. 12A-12C illustrate an exemplary deployment of an exemplary anchor having and annular spring for retaining the anchor to tissues: FIG. 12A shows a distal tip of the anchor deployed from an outer sheath; FIG. 12B shows the annular spring deployed out of the outer sheath; and FIG. 12C shows a proximal retaining hump deployed out of the outer sheath.

FIGS. 12D-12F illustrate an exemplary deployment of an exemplary anchor having and retaining humps for retaining the anchor to tissues: FIG. 12D shows a distal tip of the anchor deployed from an outer sheath; FIG. 12E shows a distal retaining hump deployed out of the outer sheath; and FIG. 12F shows a proximal retaining hump deployed out of the outer sheath.

FIGS. 13A and 13B illustrate an exemplary mitral implant system that includes a conically shaped “D-Flower” dock implant: FIG. 13A shows the dock positioned on the atrial side of the mitral valve and tethers anchored to the ventricle walls; FIG. 13B shows a replacement valve positioned within the dock.

FIGS. 14A and 14B illustrate an exemplary mitral implant system that includes a half-dome shaped dock implant: FIG. 14A shows the dock positioned on the atrial side of the mitral valve and tethers anchored to the ventricle walls; FIG. 14B shows a replacement valve positioned within the dock.

FIGS. 15A-15C illustrate an exemplary implant having a coaptation structure: FIG. 15A shows a top view of the implant; FIG. 15B shows a top perspective view of the implant; and FIG. 15C shows a side view of the implant.

FIGS. 16A-16D illustrate an exemplary implant configured to modify the size of a valve annulus: FIG. 16A shows a top perspective view of the implant; FIG. 16B shows a closeup view of one-way hooks of the implant; FIG. 16C shows a closeup side view of the implant being expanded by expansion of a balloon; and FIG. 16D shows a closeup side view of the implant being contracted by deflation of the balloon.

FIG. 17A illustrate an exemplary anchoring device configured for transmuscular anchoring; FIG. 17A shows a top perspective view of the device; and FIG. 17B shows a side view of the device.

FIGS. 17C-17F illustrate an exemplary deployment of the anchoring device of FIGS. 17A and 17B: FIG. 17C shows delivery of the anchoring device at a target site along the ventricle wall; FIG. 17D shows one side of a compression coil of the anchoring device deployed; FIG. 17E shows a washer of the anchoring device deployed; and FIG. 17E shows another side of the compression coil of the anchoring device deployed.

FIGS. 18A and 18B illustrate an exemplary anchoring device configured for intermuscular anchoring: FIG. 18A shows a section view of the anchoring device within a delivery catheter; and FIG. 18B shows a side view of the anchoring device secured within a ventricle wall.

FIGS. 18C-18E illustrate an exemplary deployment of the anchoring device of FIGS. 18A and 18B: FIG. 18C shows delivery of the anchoring device at a target site along the ventricle wall; FIG. 18D shows one side of a compression coil of the anchoring device deployed; FIG. 18E shows a washer of the anchoring device deployed.

FIGS. 19A-19C illustrate an exemplary tube having a set of expandable arms as part of an anchoring device: FIG. 19A shows a perspective view of the tube in a radially compact state; FIG. 19B shows the tube in a radially expanded state; and FIG. 19C shows the tube in a flattened state.

FIG. 19D illustrates an exemplary anchor having two sets of expandable arms for retaining the anchor to a wall of the heart.

FIGS. 20A and 20B illustrate another exemplary expandable tube as part of an anchoring device: FIG. 20A shows a perspective view of the tube in a radially compact state; and FIG. 20B shows the tube in a flattened state.

FIGS. 21A and 21B illustrate a further exemplary expandable tube as part of an anchoring device: FIG. 21A shows a perspective view of the tube in a radially compact state; and FIG. 20B shows the tube in a flattened state.

### DETAILED DESCRIPTION

The disclosure herein relates to systems, devices, and methods for reducing (e.g., mitral) valve regurgitation and/or performing valve replacement. The devices may include a valve implant configured for placement adjacent to the diseased valve. The valve implant may be configured to repair aspects of a diseased valve or may be configured to replace the diseased valve, or portions of the diseased valve. In some cases, one or more tethers may be attached to the valve implant to apply a tensioning force against the valve implant to anchor and help secure the valve implant. The tethers may be coupled to one or more anchors attached to an inner surface of the heart, such as the inner surface of the ventricle.

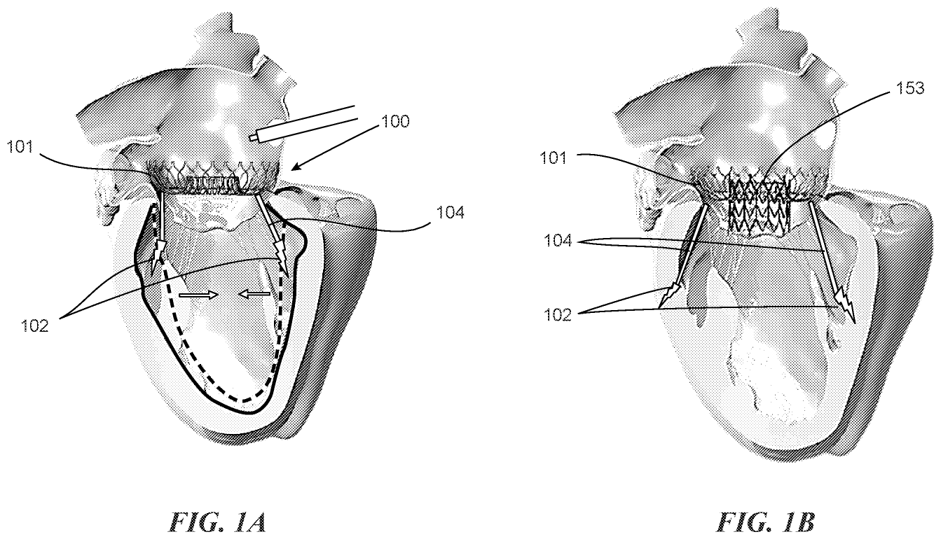

FIGS. 1A and 1B show two examples use cases for the valve implants described herein. FIG. 1A shows an implant 100 for mitral repair and/or left ventricle reverse remodeling. The implant 100 is placed adjacent to the mitral valve and includes a dock 101 positioned within the valve annulus and tethers 104 connected to anchors 102 in the ventricle wall of the heart. In this example, the dock 101 has an annular shape with an inner wall and an outer wall (e.g., half toroid shape). FIG. 1B shows a valve replacement implant 153 for mitral replacement (e.g., via TMVR). As shown in FIG. 1B, the valve replacement implant 153 may be positioned within the dock 101. The valve implant 153 may be positioned within a central opening of the dock 101 (e.g., within the inner wall).

In some cases, the mitral valve repair (FIG. 1A) may be implemented as phase

one in a treatment and the mitral valve replacement (FIG. 1B) may be implemented as phase two in the treatment. The dock implant 101 may be a supra-annular valve implant that is secured to left ventricle tethers 102. At phase one (FIG. 1A), force from supra-annular dock 101 may reduce annular dimensions to treat mitral regurgitation (MR). Tension on the tethers 104 may stabilize and may reduce left ventricle dilation (LVD). The tethers 104 may cross the valve annulus at commissures of the native valve, and may not affect native valve function. This arrangement may provide the ability to optimize aorto-mitral angulation (AMA) for later TMVR at phase two (FIG. 1B). In some cases, the dock 101 may allow a simple valve-in-ring (ViR) procedure, if necessary.

Some advantages of the dock implants 101 described herein (for use in mitral valve repair (phase 1)) may include: ability to reshape mitral annulus to reduce MR; reduced or no LVD exclusion risk; ability to optimize aorto-mitral angulation (AMA) for TMVR; easy transseptal delivery of repair dock (e.g., having a 28Fr catheter size or less); and/or the LV tethers may facilitate reverse remodeling. The tethered dock implant 101 may allow TMVr via ventriculoplasty. The tethers 104 may stabilize the left ventricle and facilitate reverse remodeling. The tethered dock implant 101 may be preset or adjusted AMA to avoid left ventricular outflow tract (LVOT) obstruction prior to valve implantation. The tethered dock implant 101 may be configured for a less than 28Fr transseptal delivery system. The tethered dock implant 101 may offer a preset valve landing zone for future TMVR, ViR-like procedure.

Some of the advantages of the valve replacement implants described herein (valve in ring (phase two)): ability for reintervention if MR returns; reduces or avoids left ventricle outflow tract (LVOT) obstruction; and/or easy transseptal delivery of transcatheter heart valve (THV) or ViR Procedure (e.g., having a 21Fr catheter size or less).

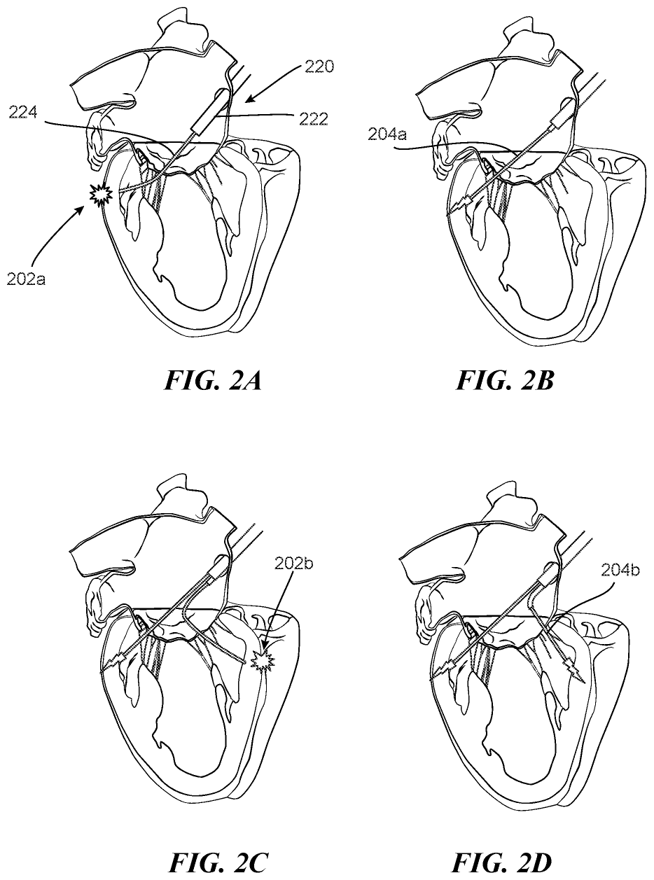

FIGS. 2A-2D illustrate an example procedural flow for anchor placement. At FIG. 2A, an anchor group having a first anchor 202a may be deployed in the ventricular wall below the anterolateral commissure. In some cases, an anchor delivery system 220, which includes a transseptal introducer 222 and a steerable sheath 224 may be used to deliver the anchor 202 to a target site. At FIG. 2B, an anterolateral anchor tether 204a may be secured proximally. At FIG. 2C, a second anchor group having a second anchor 202b may be deployed in the ventricular wall below posteromedial commissure. At FIG. 2D, a posteromedial anchor tether 204b may be secured proximally.

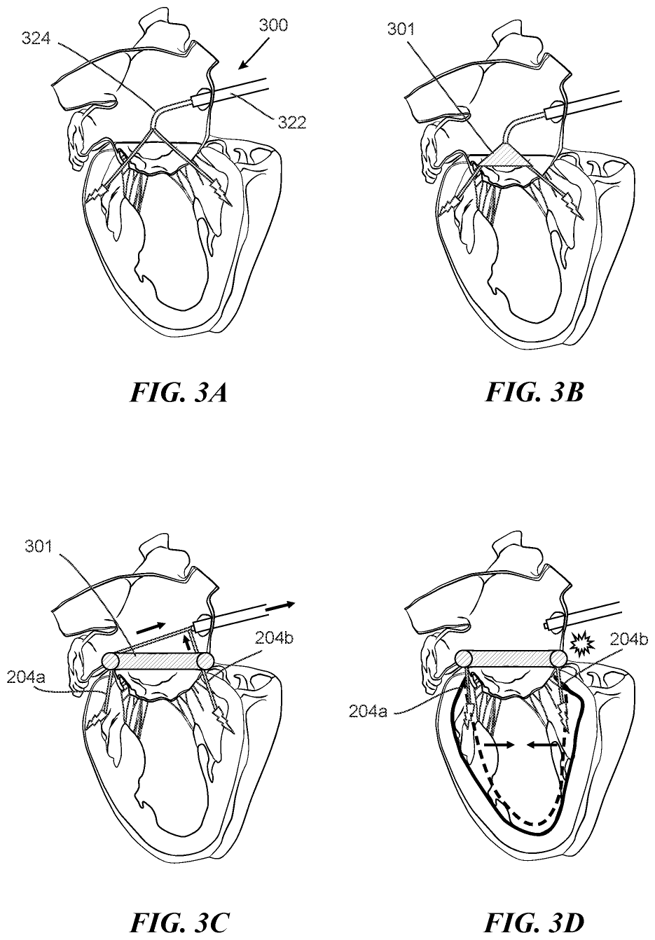

FIGS. 3A-3D illustrate an example procedural flow for dock placement. At FIG. 3A, a dock delivery system 300 may be advanced across septum and oriented centrally over mitral annulus. The delivery system 300 may include a transeptal introducer 322 and a steerable catheter 324. FIG. 3B, shows a dock 301 being deployed in the left atrium. At FIG. 3C, the tethers 204a and 204b may be tensioned to reduce LV volume and seat the dock 301 on the mitral annulus. At FIG. 3D, the tethers 204a and 204b may be detached, and the dock 301 may be released from delivery system 300. In some cases, the procedure may optionally proceed with a ViR-TMVR procedure.

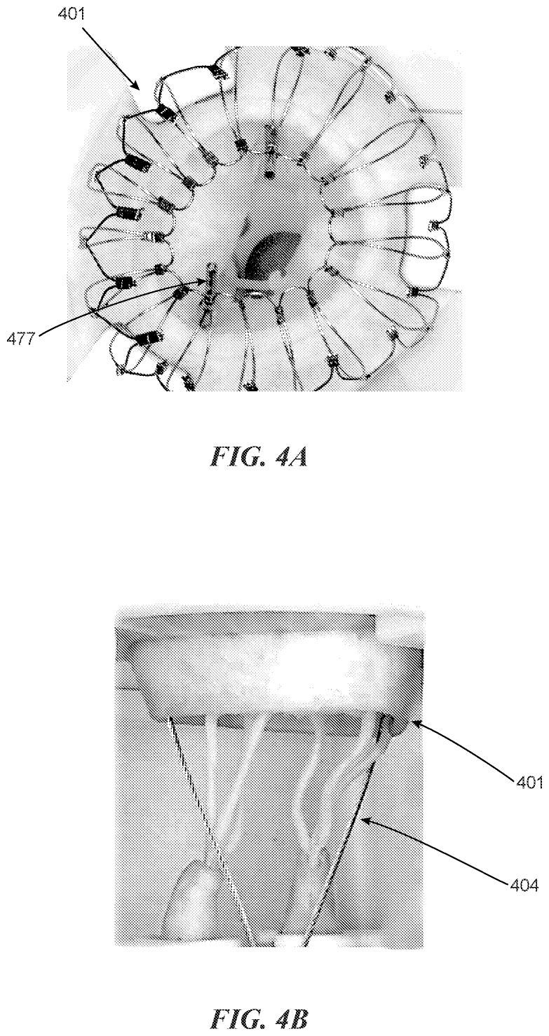

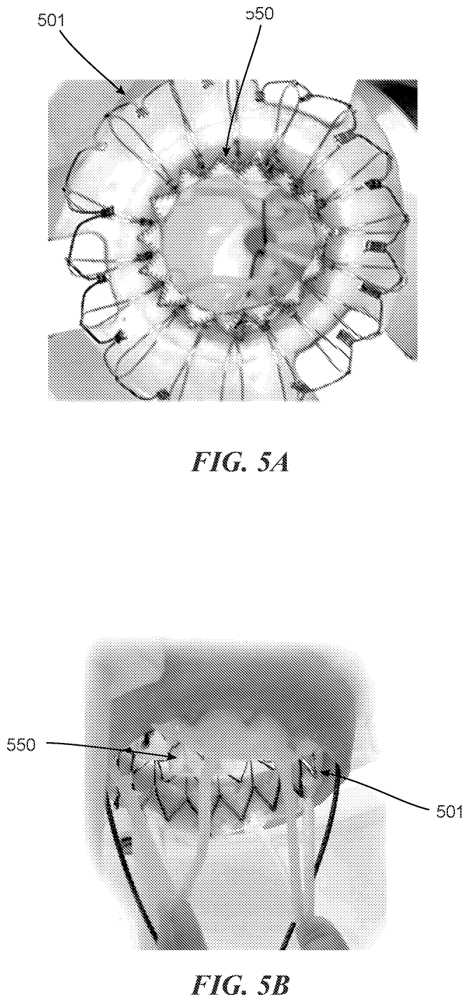

FIGS. 4A and 4B illustrate an example dock 401 that may be configured for mitral repair and/or left ventricle reverse remodeling (phase one) treatment. FIG. 4A shows at top-down view of the dock 401 as it would be supported in an atrium. As shown, the dock 401 may have a simplified circular geometry. In this example, the dock 401 includes tether connectors 477 on opposite sides of the dock 401, which are configured to couple with the tethers 404. FIG. 4B shows a side view of the ventricle side of the dock 401 illustrating the tethers 404 connected to the valve implant and directed toward the left ventricle wall for remodeling the ventricle. As shown, the dock 401 and tethers 402 may leave space for the native leaflets to be unobstructed.

FIGS. 5A and 5B illustrate an example mitral valve implant 550 inserted with in a dock 501 (e.g., similar to the dock of FIGS. 4A and 4B). FIG. 5A shows a top-down view of the mitral valve implant 550 deployed in the dock 501. FIG. 5B shows a perspective view of the mitral valve implant 550 deployed in the dock 501. The mitral valve implant 550 may be configured to open the native valve leaflets. The mitral valve implant 550 may apply a radially outward force against the dock 501, with limited to no outward radial force applied to the atrioventricular junction.

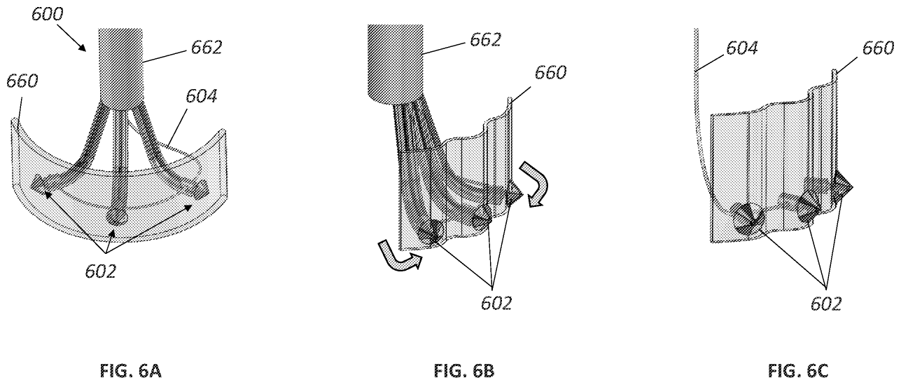

FIGS. 6A-6C illustrate an example deployment of an example anchoring device 600 for anchoring one or more tethers to the left ventricle wall 660. The anchoring device 600 may include a group of anchors, as shown. At FIG. 6A, the anchoring device 600 may be delivered to a target site along the ventricle wall 660 via a scaffold catheter 662, where a group of anchors 602 are deployed to the ventricle wall 660. At FIG. 6B, the anchors 602 may be circumferentially cinched to reduce the local perimeter of the ventricle wall 660. This may be done, for example, by placing tension on one or more tethers 604 that are coupled to the anchor s 602. At FIG. 6C, the anchors 604 may be released from the scaffold catheter 662 leaving behind the anchor 602 and tether 604 secured to the ventricle wall 660, where the other side of the tether 604 is secured to the dock at the mitral valve. The tension on the tether 604 provide a force that reduces or maintains a ventricle volume.

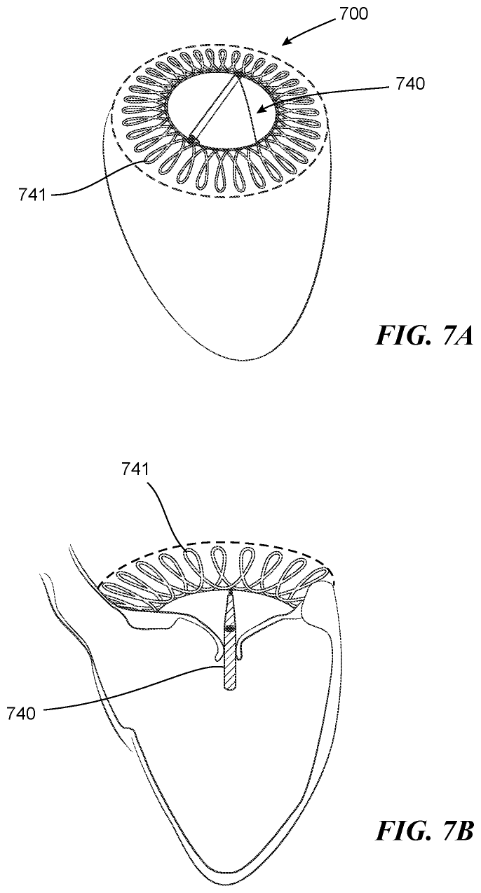

FIGS. 7A and 7B illustrate an example of a valve implant 700 that can act as a dock for an anchor device and that may also include a coaptation blocker 740 (e.g., also referred to as coaptation structure or mating body) for coaptation with native leaflets of the valve. The valve implant 700 may include frame 741 (dashed annular portion) (e.g., also referred to as an annular component or scaffold) that may be configured to anchor to the annulus of the valve implant 700 via tethers that are attached to the LV wall. A flexible, compliant, non-thrombogenic coaptation blocker 740 may be positioned at a central portion within annular component 741. The blocker 740 may provide a coaptation surface in between the native leaflets of the native valve. The coaptation blocker 740 may provide an additional mechanism to resolve MR in conjunction with ‘reining in’ of the LV wall to reduce chordal tension. In some cases, the coaptation blocker 740 may be designed so as to be pushed out of the way if a valve replacement is later performed. Alternatively, one side of the coaptation blocker 740 may be cut to accommodate the valve replacement. In some cases, the coaptation blocker 740 may include a fluid-filled polymer, or an ePTFE-covered nitinol structure that has sufficient compliance to accommodate variations in leaflet shape, length, curvature along the line of coaptation, etc.

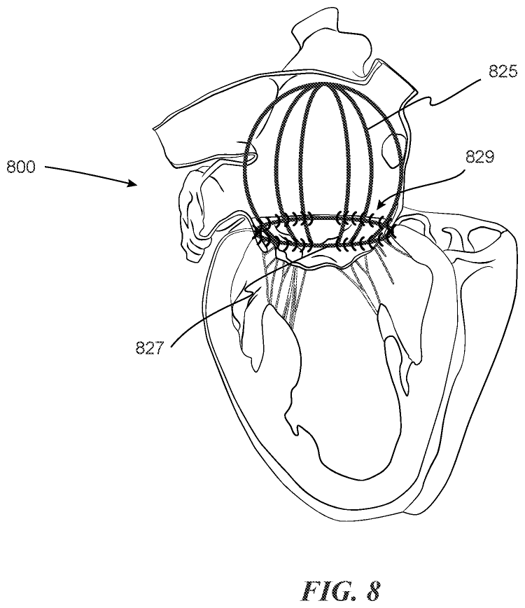

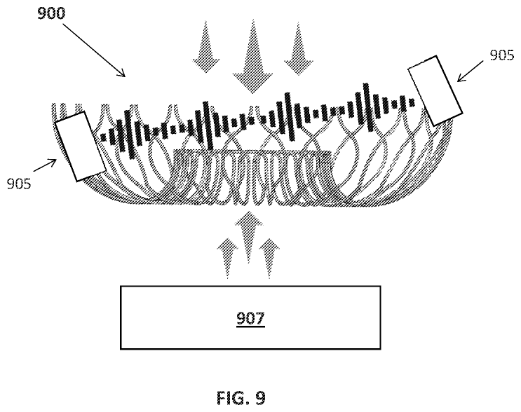

FIG. 8 illustrates an example valve implant 800 having features for atrial deployment anchoring (e.g., in place of or in addition to the anchored tethers described herein). A supra-annular cage 825 and an annular ring 827 may be configured for anchoring to a valve annulus. The valve implant 800 may include one or more elements for implantation into tissue 829. Force from the ring 827 may reduce annular dimensions to treat mitral regurgitation (MR). The ring 827 may later accommodate a prosthetic valve for later TMVR. In some cases, the valve implant 800 may include self-expanding structures (e.g., formed of an alloy comprising nickel and titanium). In some cases, the valve implant 800 may be an expandable, e.g., by force applied from a balloon. The expandable implant 800 may be formed, for example, of a cobalt chromium material or similar material.

FIG. 9 illustrates an example valve implant 900 (e.g., similar to the dock implants described herein) that may be used for in vivo diagnostics. The valve implant 900 may provide a support structure for inductively coupled transonic flow meter sensors 905. For example, the sensors 905 may be configured to send and/or receive signals from/to a transonic transducer and/or receiver 907. The valve implant 900 may have a low delivery profile with sufficient circumferential area for sensor positioning. Such arrangement may be useful in conjunction with diagnostic tools for characterizing mitral regurgitation (MR) progression after repair. The sensors 905 may be positioned around a periphery of the implant 900.

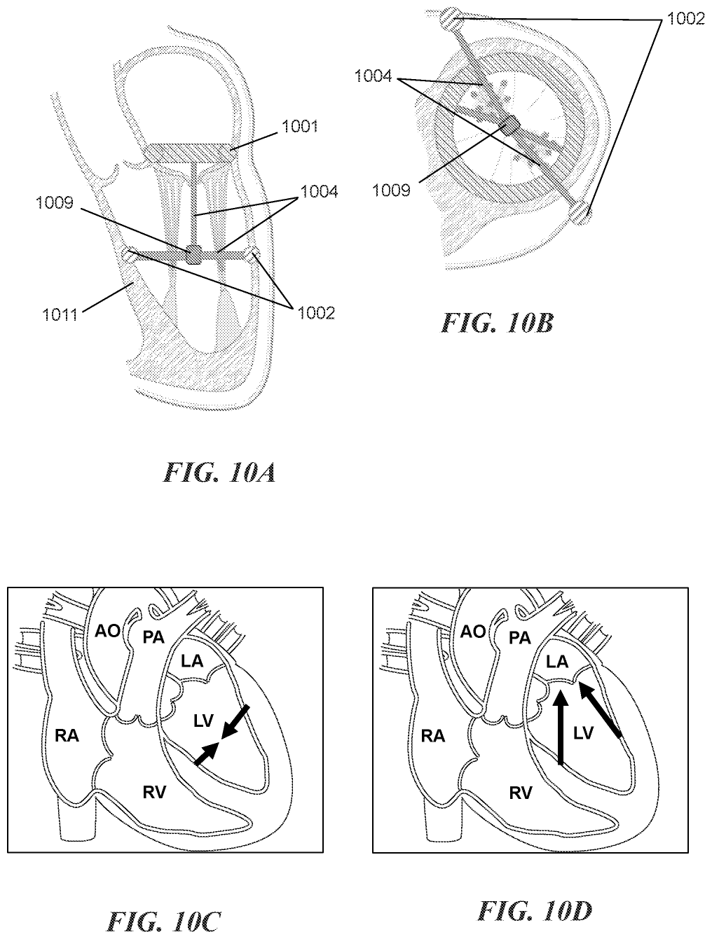

FIGS. 10A and 10B illustrate an alternative example of a tethered transeptal TMVR dock implant 1001 that allows for cross ventricle anchoring. FIG. 10A shows a cross section commissural view and FIG. 10B shows a bottom view (with the dock 1001 above valve annulus). The dock 1001 may passively sit above the valve annulus under tension from LV anchors 1002. The dock 1001 may be self-expanding (e.g., made of a shape memory material (e.g., nitinol)). LV muscle anchors 1002 may be configured for lateral deployment along the ventricle wall 1011. A tensioning nut 1009 may allow lateral dimensional reduction of LV with a greater proportional reduction in LV volume for prescribed tether 1004 displacement. This is illustrated in FIG. 10C (with the LV/papillary anchors muscle anchors 1002) versus FIG. 10D (without the LV/papillary muscle anchors 1002). The LV anchors 1002 may be configured not to interfere with the conduction system. The LV tethers 1004 may allow controllable LV volume reduction pre-TMVR. The TMVR procedure may be simplified to a VIR approach.

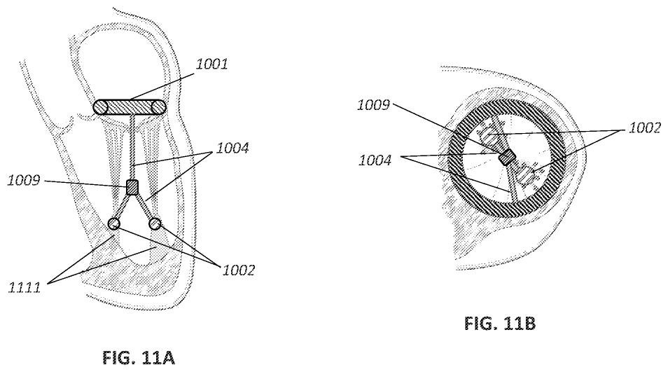

FIGS. 11A and 11B illustrate a variation of the dock implant of FIGS. 10A-10D where the LV anchors 1102 are configured to secure to papillary muscles 1111. FIG. 11A shows a cross section commissural view and FIG. 11B shows a bottom view (with the dock 1101 above valve annulus). This arrangement may provide a surgical repair-like approach to improve leaflet tethering from a dilated ventricle.

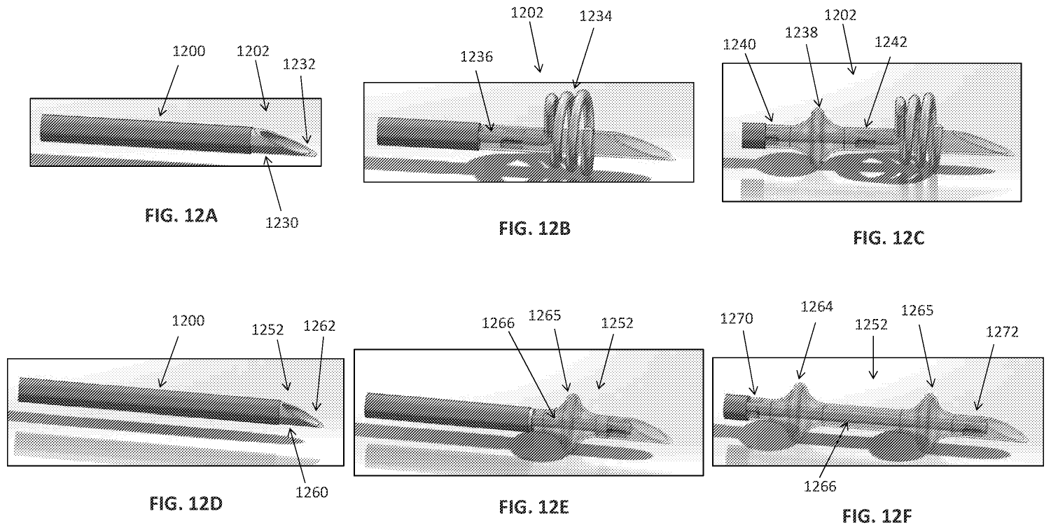

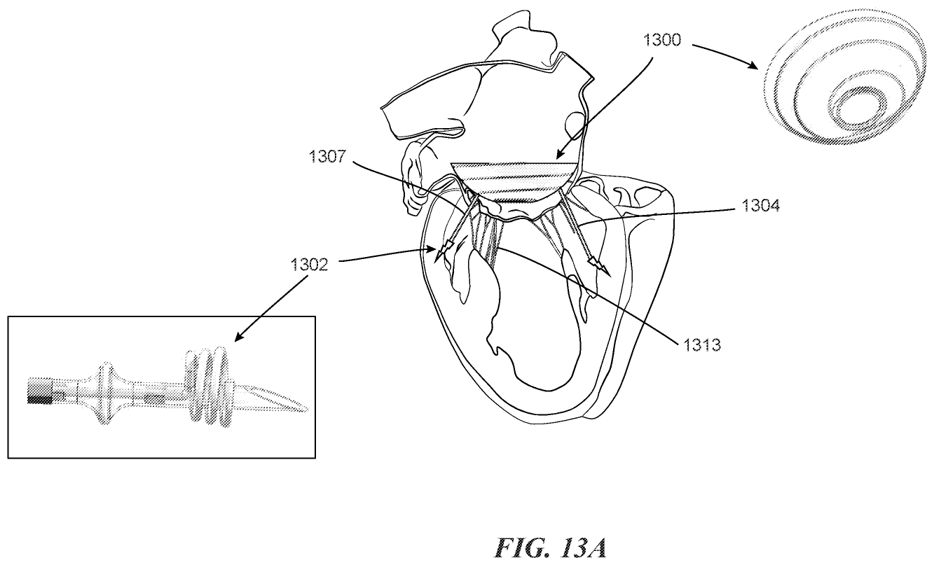

FIGS. 12A-12F illustrate two different ventricular anchors having retaining features for engaging with tissue of the ventricle wall. The retaining features may include a first expandable portion configured for placement outside the ventricle and a second expandable portion configured for placement inside the ventricle. In some cases, at least some portions the anchors in FIGS. 12A-12F may be made of a shape memory alloy (e.g., nitinol).

FIGS. 12A-12C show deployment of one example anchor 1202 from an outer sheath 1200. FIG. 12A shows the anchor retracted within the outer sheath 1200 except for a sharp distal end of the anchor. The anchor may include a hypotube 1230 (e.g., laser cut hypotube) having an elongated shape while in the retracted state with the sharp distal tip 1232 exposed for puncturing through the ventricle wall. FIG. 12B shows the anchor after the outer sheath 1200 is pulled proximally (or the anchor 1202 is pushed distally) to expose a first expandable portion of the anchor 1202 having an annular spring structure 1234. The annular spring 1234 may be part of a wire 1236 that is positioned within the hypotube 1230. The wire 1236 may be made of a shape memory alloy and have a distal end that is shape-set in the coiled annular shape, as shown. The wire 1236 can take on a straight shape when retracted within the hypotube 1230. The wire 1236 can be pushed distally through an opening of the hypotube 1230 to release the distal end of the wire 1236 such that the wire 1236 takes on a coiled annular shape outside of the hypotube 1230, as shown.

FIG. 12C shows the outer sheath 1200 pulled further proximally (or the anchor 1202 pushed further distally) to expose a second expandable portion of the anchor 1202 having a retaining hump 1238 proximal to the annular spring 1234. The retaining hump 1238 may correspond to a shape-set strut formed in the hypotube 1230 of the anchor 1202. When the anchor 1202 is within the outer sheath 1200, the retaining humped 1238 may expand longitudinally and take on an elongated shape having a smaller diameter. When the retaining hump 1238 is released from the outer sheath 1200, the retaining hump 1238 can return to an expanded hump shape, as shown, by foreshortening the hypotube 1230. Once inserted within the ventricle wall, the annular spring 1234 may be configured to sit on the outside of the ventricle wall and the retaining hump 1238 may be configured to sit on the inside of the ventricle wall. When expanded, the retaining hump 1238 may foreshorten and apply a compression force against the annular spring 1234, thereby trapping the ventricle wall therebetween. The wire 1236 and hypotube 1230 may include locking features to lock the anchor 1202 to the ventricle wall. In this example, the wire 1236 may include detent cutouts that engage with corresponding (e.g., laser cut) tabs of the hypotube 1230. The anchor 1202 may include two locking features: a distal locking feature 1242 on a distal side of the retaining hump, and a proximal locking feature 1240 on a proximal side of the retaining hump. The foreshortening of the hypotube 1230 during deployment can result in longitudinal displacement of the hypotube 1230 relative to the inner wire 1236 and cause the detent cutouts of the wire 1236 to engage with the corresponding tabs of the hypotube 1230. This interlock can prevent the hypotube 1230 from elongating, which can reduce the diameter of the retaining hump 1238 and possibly crossing back into the ventricle wall (e.g., effectively causing the anchor 1202 to fall out of the ventricle wall). In some examples, this locking feature may be configured to permanently lock the anchor 1202 to the ventricle wall. The inherent foreshortening of the retaining hump 1238 may also cause chronic axial compression along the hypotube 1230 axis to help ensure hemostasis at the puncture site.

FIGS. 12D-12F show deployment of another example anchor 1252, in this case, having two retaining humps 1264 and 1266. FIG. 12D shows the anchor 2152 retracted within the outer sheath 1200 except for a pointed distal end 1262 of the anchor 1252. FIG. 12E shows the anchor 1252 after the outer sheath 1200 is pulled proximally (or the anchor 1252 is pushed distally) to expose a distal retaining hump 1265 of the hypotube 1260. FIG. 12F shows the outer sheath 1200 pulled further proximally (or the anchor 1252 pushed further distally) to expose a portion of the anchor 1252 having a proximal retaining hump 1264. The anchor 1252 may include a proximal locking feature 1270 proximally located with respect to the proximal retaining hump 1264 and a distal locking feature 1272 distally located with respect to the distal retaining hump 1265. Each of the proximal 1270 and distal 1272 locking features may include a detent cutout of the wire 1236 that is configured to engage with a corresponding tab of the hypotube 1260.

Deployment of the anchor 1252 in FIGS. 12D-12F may involve puncturing through the ventricle wall using the sharp tip, unsheathing the distal length of anchor 1252 so that the distal retaining hump 1265 expands outside of the ventricle wall and cannot cross back proximally into the ventricle. The anchor 1252 may then further be deployed exposing the proximal retaining hump 1264 within the ventricle on the opposing side of the ventricle wall as the distal retaining hump 1265. The inherent foreshortening of the proximal hump 2164 can result in a compression force toward the distal hump 1265. Likewise, the inherent foreshortening of the distal hump 1265 can result in a compression force toward the proximal hump 1264, thereby trapping the ventricle therebetween and securing the anchor 1252 to the ventricle wall. The inherent foreshortening of the proximal 1264 and distal 1265 retaining humps can also cause chronic axial compression along the tube axis to help ensure hemostasis at the puncture site. The foreshortening of the hypotube 1260 during deployment can result in longitudinal displacement of the hypotube 1260 relative to an inner wire 1266, thereby causing the detent cutouts of the wire 1266 to engage with the corresponding tabs of the hypotube 1260.

Note that the mechanisms for using the anchors 1202 and 1252 shown in FIGS. 12A-12F may be a corollary to a shape change of an amplatzer plug being exposed from an outer sheath 1200.

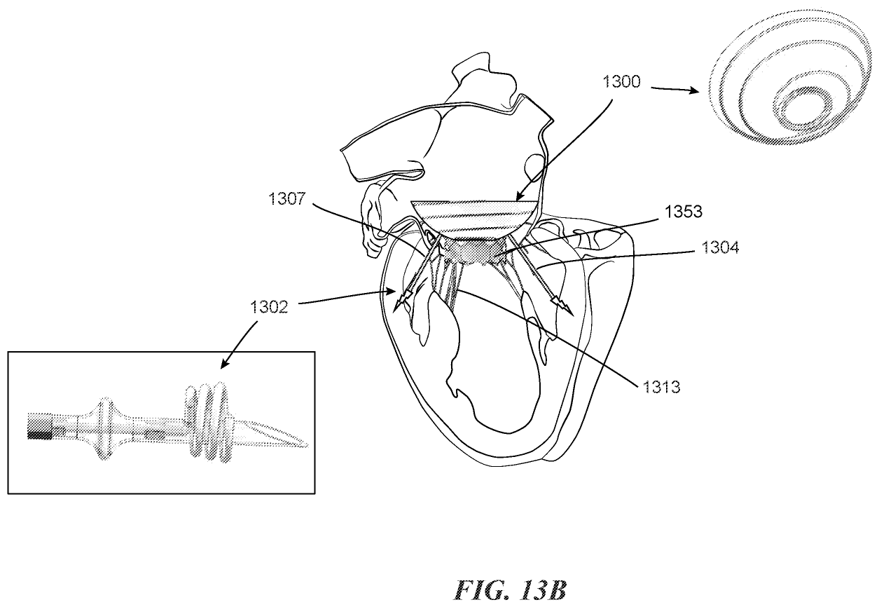

FIGS. 13A and 13B illustrate another example mitral implant system. FIG. 13A shows a conically shaped “D-Flower” dock 1300 positioned on the atrial side of the mitral valve and tethered via tethers 1304 to anchors 1302 secured along the ventricle wall. A tensioning nut 1307 can be configured to adjust the aorto-mitral angulation (AMA) for later replacement valve implantation at phase two. The chordae tendineae 1313 may allow for adjustable length. The lateral tension provided by the anchored tethers 1307 may provide greater volume reduction. FIG. 13B shows the system of FIG. 13A with the replacement valve 1353 positioned within the annulus of the dock implant 1300. The anchored tethers 1307 may have a fixed length.

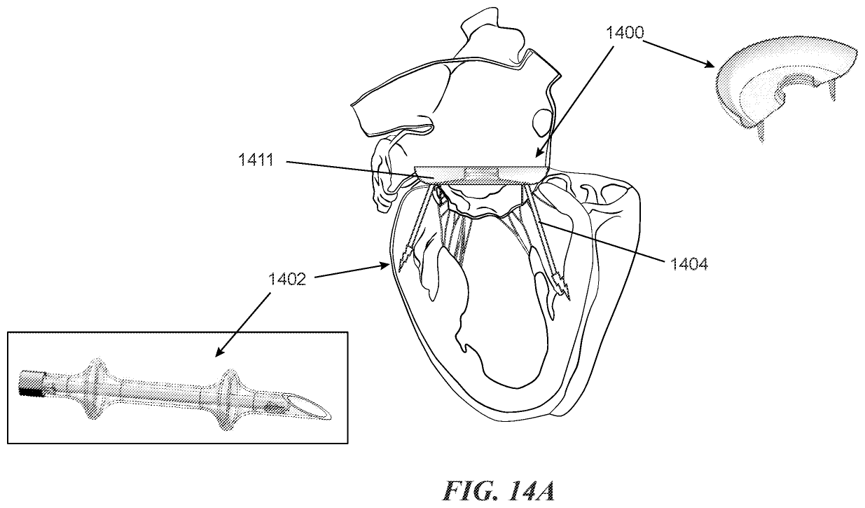

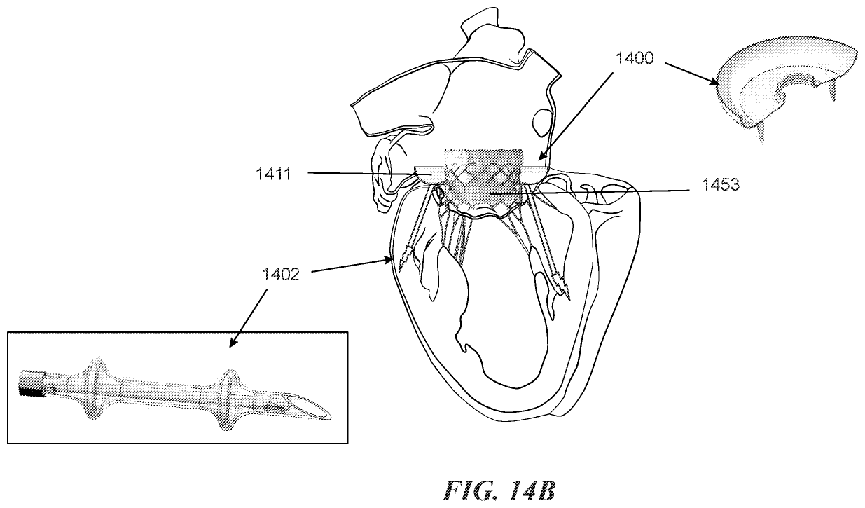

FIGS. 14A and 14B illustrate another example mitral implant system. FIG. 14A shows a half-dome shaped dock implant 1400 positioned on the atrial side of the mitral valve and tethered via tethers 1404 to anchors 1402 secured along the ventricle wall. In some cases, the half-dome shaped dock implant 1400 undergoes a two-step heat treatment. Such two-step heat treatment may be used to achieve a complex geometry with tight curvatures and inversion of tubular structures. In some cases, multiple heat treatments are used to make sure that a shape memory material (e.g., nitinol) of the dock implant 1400 is not strained or bent past its material limits. A suture clasp 1411 can be configured to hold tension applied. The tethers 1404 may be secured at commissural regions of the native valve to enable coaptation and access native leaflet functionality. This may allow the ability to optimize AMA. The lateral tension provided by the tethers 1404 can provide greater volume reduction. FIG. 14B shows the system of FIG. 14A with the replacement valve 1453 positioned within the annulus of the dock implant.

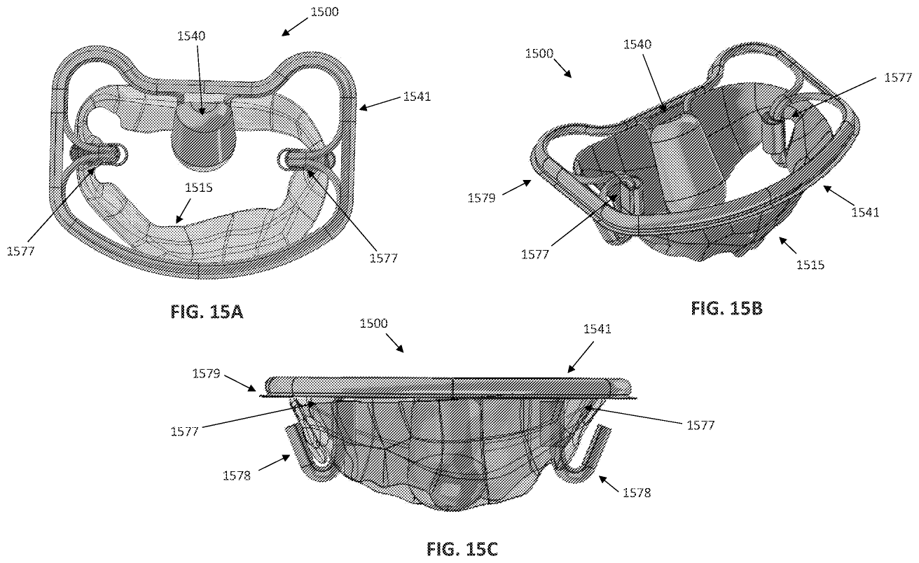

FIGS. 15A-15C illustrate another example implant 1500 (e.g., dock) having a coaptation structure 1540 that is configured to aid functioning of valve leaflets, similar to the valve implant of FIG. 7. The implant 1500 may include a frame 1541 configured to expand and anchor the implant 1500 within the valve opening. In some cases, the frame 1541 may be configured for positioning within the atrium (e.g., left atrium), which may prevent migration of the implant 1500 into the ventricle (e.g., left ventricle) and provide an oppositional force against tension from tethers (not shown) that may be attached to the frame 1541 and anchored in the ventricle (e.g., left ventricle).

The implant 1500 may include a coaptation structure 1540 configured for positioning at the level of native leaflet coaptation, such that the native leaflets 1515 can coapt against it, thereby preventing MR. The coaptation structure 1540 may extend radially inward within the opening of the frame 1541 to position the coaptation structure 1540 between the native leaflets 1515. The coaptation structure 1540 may be designed such that it is flexible enough so that the native leaflets 1515 can move it to the point of normal leaflet coaptation. The coaptation structure 1540 may be located at any point along the line of coaptation of the native leaflets 1515. The surfaces of the coaptation structure 1540 can improve closure of the native leaflets 1515, which may improve heart function. For example, improving heart function can comprise reducing mitral regurgitation. In some cases, the coaptation structure 1540 is arranged for positioning proximal to the posterior middle leaflet (P2). Such position may be preferred for the anterior leaflet engagement. The implant 1500 may work passively to aid in coaptation with the native leaflets 1515 and may be substantially non-thrombogenic.

In some cases, the implant 1500 may have different use conditions. For example, an initial use condition may involve positioning the implant 1500 within the diseased valve with the coaptation structure 1540 so that the coaptation structure 1540 may aid the native leaflets 1515 to reduce or eliminate MR. A second use condition may involve replacing the coaptation structure 1540 with a new valve prosthesis (e.g., at a later time) so that the frame 1541 circularizes the new prothesis. Additionally, the implant 1500 may be anchored in place, e.g., using any of anchor devices described herein.

The anchor 1500 may further include anchoring tabs 1577 that may be configured to anchor the implant 1500 within the valve annulus. The anchoring tabs 1577 may extend from the frame 1541 radially inward within the opening of the frame 1541. In some cases, the anchoring tabs 1577 may be arranged for positioning within or near the medial and lateral commissures, which may prevent or reduce leakage at the medial and/or lateral commissures. The anchoring tabs 1577 may have hook portions 1578 configured to wrap around and/or pass through the leaflets on a ventricle side of the implant 1500. In some cases, the hook portions 1578 may be configured to couple with tethers coupled to an anchor in the left ventricle, as described herein. The implant 1500 may be tethered to any of the anchor devices described herein.

In some examples, the implant 1500 may include a thin lip 1579 around a perimeter of the frame 1541. This arrangement may promote ingrowth of tissue onto the lip 1579 and the frame 1541.

In some cases, at least a portion of the implant 1500 may include a material that promotes ingrowth of tissues. For example, the implant 1500 may include a fabric and/or polymer (e.g., polytetrafluoroethylene) material configured to promote tissue growth. In some cases, the implant includes a tissue growth promoting covering and/or coating.

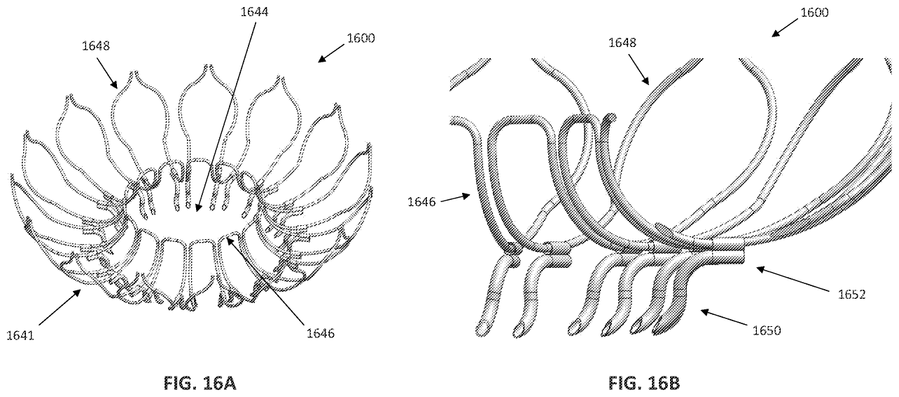

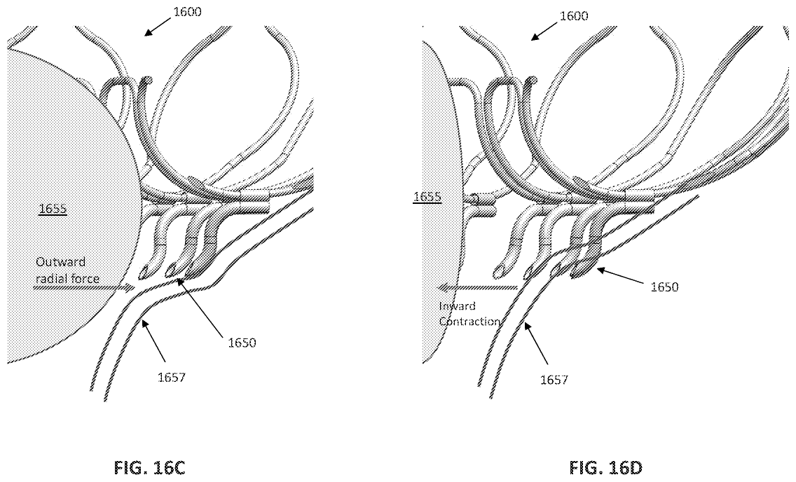

FIGS. 16A-16D illustrate an example implant 1600 (e.g., dock) configured to modify the size of a valve annulus. The implant 1600 may include a frame 1641 made of a shape-memory material, such as nitinol wire. The implant 1600 may have an annular shape with a central opening 1644 defined by a circumference of an internal wall 1646. The central opening 1644 may be undersized relative to the valve annulus when the implant 1600 is in a “default” low energy state. When positioned within the valve annulus, the smaller sized central opening 1644 may function as the effective valve annulus and improve leaflet coaptation. In some cases, the central opening 1644 may have a diameter relative to the diameter of the valve annulus that is within a range bounded by any two of the following values: 5%, 10%, 20%, 30%, 40%, 50%, 60%, 70%, 80%, 90% and 95%.

An atrial side of the implant 1800 may include a series of outwardly radiating petals 1648 to provide structural stiffness. A ventricle side of the implant 1800 may include a series of inwardly radiating one-way hooks 1650 that are configured to engage with tissue 1657 and secure the implant 1800 in place.

FIGS. 16C and 16D illustrate the use of a balloon 1655 to deploy the implant 1600 within the native valve. The balloon 1655 may be positioned within the central opening 1644 of the implant 1600 and inflated, as shown in FIG. 16C. The outward radial force from the expansion of the balloon 1655 may engage with the frame 1641 and press the one-way hooks 1650 against the valve annulus tissue 1657. The one-way hooks 1650 may have inwardly curved shapes that allow entry of the hooks 1650 into the valve annulus tissue 1657. In some cases, the balloon 1655 may have a spherical shape that may temporarily occlude blood flow through the valve. In some cases, the balloon 1655 may have one or more opening to allow blood to flow therethrough and through the valve. For example, the balloon 1655 may have a toroid shape having a central opening for blood to flow through the valve.

Once the one-way hooks 1650 are sufficiently engaged with the tissue 1657, the balloon 1655 may be deflated to retract the balloon 1655 an remove the radially outward force on the frame 1641. Due to their shapes, the inwardly curved hooks 1650 may remain engaged within the tissue 1657, thereby securing the implant 1600 within the valve.

In some cases, at least a portion of the implant 1600 may include a material that promotes ingrowth of tissues. For example, the implant 1600 may include a fabric and/or polymer (e.g., polytetrafluoroethylene) material configured to promote tissue growth. In some cases, the implant includes a tissue growth promoting covering and/or coating.

The implant 1600 may be tethered to any of the anchor devices described herein. Tethers for coupling the implant 1600 to the anchor maybe be coupled to the frame 1641 at any of a number of locations. In some cases, the tethers are coupled at or near a junction region 1652 where the one-way hooks 1650 are attached to the frame 1641.

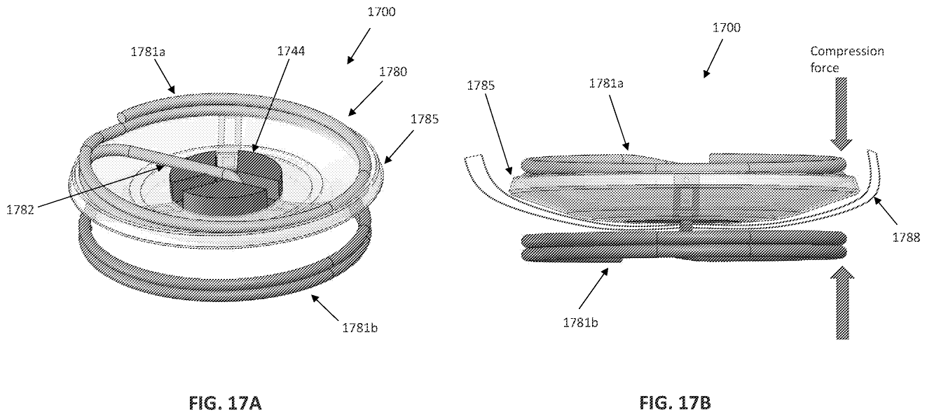

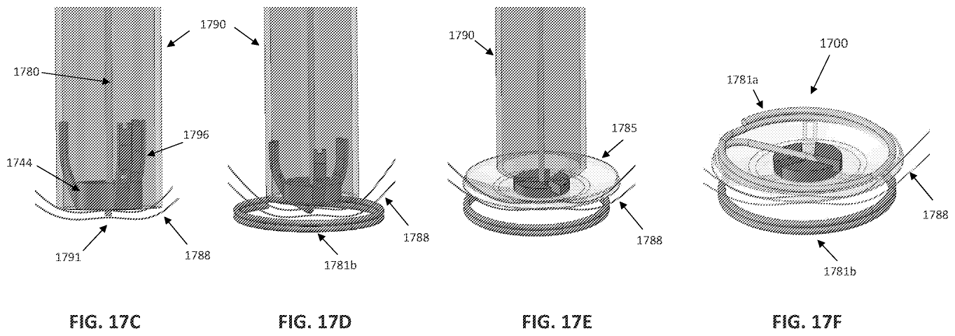

FIGS. 17A and 17B illustrate an example anchoring device 1700 configured for transmuscular anchoring. The anchoring device 1700 may be configured for anchoring through the ventricle (e.g., left ventricle) wall 1788 of the heart and may be configured to transition from a straightened delivery configuration to an expanded deployed configuration. The anchoring device 1700 in the expanded configuration may include a compression coil 1780 (e.g., wire) having a first annular portion 1781a connected to a second annular portion 1781b via a connection region 1782. Each of the first and second portions 1781a and 1781b of the compression coil 1780 may have a coiled annular shape. The connection region 1782 between first and second annular portions 1781a and 1781b of the compression coil 1780 may be positioned in central region of the device 1700. The connection region 1782 of the compression coil 1780 may be configured to pass through a ventricle wall 1788 such that the first annular portion 1781a is on one side of the ventricle wall 1788 and the second annular portion 1781b is on an opposing second side of the ventricle wall 1788. For example, the first portion 1781a may be configured for placement inside of the ventricle (e.g., left ventricle) and the second portion 1781b may be configured to placement outside of the ventricle (e.g., left ventricle).

The first and second portions 1781a and 1781b of the compression coil 1780 may be configured to apply a clamping force against the ventricle wall 1788 to secure the anchoring device 1700 to the ventricle wall 1788. The compression coil 1780 may act as a spring and be made of an elastic material capable of storing mechanical energy. The compression coil 1780 may be biased such that the first and second portions 1781a and 1781b apply the clamping force therebetween. In some cases, the compression coil 1780 may be made of a shape-memory material (e.g., nitinol). The compression coil 1780 may be in the form of a wire, where each of the first and second portions 1781a and 1781b of the compression coil 1780 is wound into a ring shape. The diameter of the wire may vary depending on a desired compression stiffness.

A washer 1785 (e.g., annular washer) may be positioned between the first and second portions of the compression coil and be configured to distribute the load from opposing sides of the compressions coil. When deployed within the ventricle wall 1788, as shown in FIG. 17B, the washer 1785 may distribute the clamping force applied by the first and second portions of the compression coil across the ventricle wall 1788. For example, the first portion of the compression coil may deliver a force against one side (e.g., proximal side) of the washer 1785, thereby compressing the washer 1785 against tissue. In addition, the second portion of the compression coil may deliver a force against the ventricle wall 1788 and the opposite side (e.g., distal side) of the washer 1785 with the heart tissue therebetween, thereby clamping the device 1700 to the heart muscle. Such clamping force may act as a compression force that is sufficient to reduce or prevent bleeding from puncturing of the ventricle wall 1788, thereby providing homeostasis. The clamping/compressive force may also allow the anchoring device 1700 to have good contact with the tissue, thereby promoting ingrowth of tissue and further securing the anchoring device 1700 to the ventricle wall 1788. In some cases, the washer 1785 has a convex surface (e.g., on a distal side of the washer 1785) that contacts the heart tissue. In some cases, the washer 1785 has a curved discus shape.

In some cases, at least a portion of the anchoring device 1700 may include a material that promotes ingrowth of tissues. For example, the device 1700 may include a fabric and/or polymer (e.g., polytetrafluoroethylene) material configured to promote tissue growth. In some cases, the device 1700 includes a tissue growth promoting covering and/or coating.

FIGS. 17C-17F illustrate an example deployment of the anchoring device 1700. At FIG. 17C, the compression coil, in the straightened delivery configuration, may be delivered to the ventricle wall 1788 via a delivery catheter 1790. In some applications, the target anchoring location is at the apical portion of the ventricle. The central support 1744 may be used to guide the compression coil within the delivery catheter 1790. In some cases, central support 1744 includes centering arms 1796 that center the central support 1744 and the compression coil 1780 within the lumen of the delivery catheter 1790. Once delivered to the anchoring site of the ventricle wall 1788, the compression coil 1780 may be advanced to puncture through the ventricle wall 1788 and outside of the heart. FIG. 17C shows a distal tip 1791 of the compression coil 1780 puncturing through tissue of the ventricle wall 1788.

At FIG. 17D, the compression coil 1780 can be further advanced until the second annular portion 1781b of the compression coil 1780 is deployed outside of the ventricle wall 1788. In some cases, a tug test is performed to confirm that the second portion 1781b of the compression coil 1780 is properly deployed and sufficiently secures the device to the ventricle wall 1788. The tug test may involve applying a pulling force to apply tension to the compression coil 1780. If it is determined that the compression coil 1780 is not sufficiently secured, the compression coil 1780 may be repositioned/redeployed until properly secured or the deployment may be abandoned.

Once the second portion 1781b of the compression coil 1780 is determined to be sufficiently deployed, the washer 1785 may be delivered through the delivery catheter 1790 and positioned over the anchoring site as shown in FIG. 17E. In some cases, the washer 1785 is made of a flexible material so that it may be delivered through the delivery catheter 1790 in a collapsed state and expanded at the anchoring site. The first portion 1781a of the compression coil 1780 may then be delivered over the washer 1785 until the device 1700 is fully deployed as shown in FIG. 17F. In some cases, one or more tethers may be coupled to the device 1700, where the one or more tethers are couple to an implant adjacent to the heart valve, as described herein.

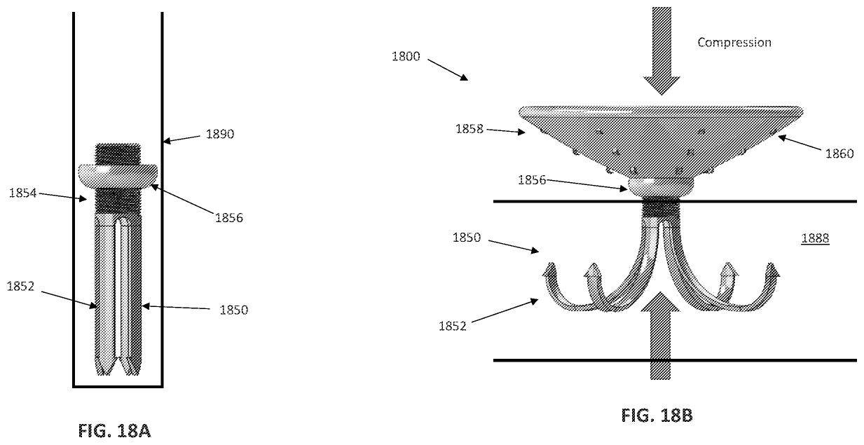

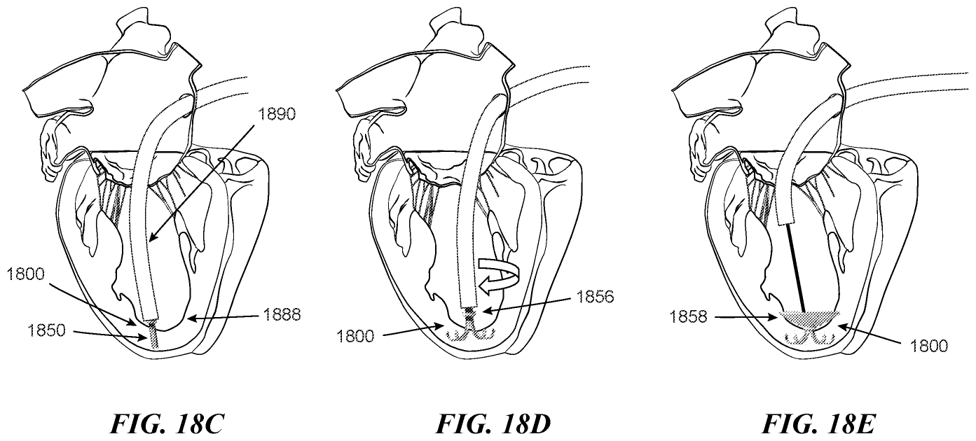

FIGS. 18A and 18B illustrate an example anchoring device 1800 configured for intermuscular anchoring. FIG. 18A shows a harpoon anchor 1850 (elongate body) portion of the device 1800 in an undeployed state, where the harpoon anchor 1850 is sheathed within a delivery catheter 1890. FIG. 18B shows the device 1800 in a deployed state secured to the wall 1888 of the ventricle (e.g., left ventricle). The harpoon anchor 1850 having wings 1852 that are configured to transition from an elongated delivery state to an expanded deployed state. A proximal side of the harpoon anchor 1850 may include a threaded portion 1854 that is configured to accept a correspondingly threaded cinching nut 1856. The wings 1852 of the harpoon anchor 1850 may be configured expand upon rotation of the cinching nut 1856. The wings 1852 of the harpoon anchor 1850 may have pointed (e.g., sharp) distal ends that are configured for tissue penetration. Once the harpoon anchor 1850 is expanded within the tissue of the ventricle wall 1888, a flange 1858 may be advanced distally (e.g., to the cinching nut 1858). The flange 1858 may have a threaded opening for winding onto the threaded portion of the harpoon anchor 1850. The flange 1858 may have a greater diameter than the threaded portion 1854 of the harpoon anchor 1850, thereby provide an efficient compression force onto the ventricle wall 1888 that is sufficient to reduce or prevent bleeding from puncturing of the ventricle wall 1888, and thereby providing homeostasis. In some cases, the flange 1858 may have a tapered (e.g., conical) shape in accordance with an apical region of the ventricle. In some cases, the flange 1858 may include protruding features 1860 that are configured to engage with tissue to secure the flange 1858 to the ventricle wall 1888. In some examples, the protruding features 1860 are hooks that are configured to hook and hold the tissue.

FIGS. 18C-18E illustrate an example deployment of the anchoring device 1800. At FIG. 18C, the device 1800 is delivered in an undeployed state to a target anchoring site on the ventricle wall 1888 via delivery catheter 1890. As with anchoring device 1700, the anchoring device 1800 may be configured for anchoring at the apical portion of the ventricle. The harpoon anchor 1850 of the device 1800, in an undeployed state, may then be advanced to puncture the tissue of the ventricle wall 1888. At 18D, the cinching nut 1856 may be rotated (e.g., by rotating the catheter) to cause the harpoon anchor 1850 to expand into the tissue, thereby securing the device 1800 within the ventricle wall 1888. A tug test may be performed to confirm that the device 1800 is sufficiently secured to the ventricle wall 1888. At 18E, the flange 1858 may be advanced through the delivery catheter 1890 and positioned on over the inside surface of the ventricle wall 1888, in this case, in the apical region of the ventricle. The compressive force provided by the flange 1858 may provide good contact with the tissue, thereby promoting ingrowth of tissue and further securing the anchoring device 1800 to the ventricle wall 1888. In some cases, one or more tethers may be coupled to the device 1800, where the one or more tethers are couple to an implant adjacent to the heart valve, as described herein.

In some cases, at least a portion of the anchoring device 1800 may include a material that promotes ingrowth of tissues. For example, the device 1800 may include a fabric and/or polymer (e.g., polytetrafluoroethylene) material configured to promote tissue growth. In some cases, the device 1800 includes a tissue growth promoting covering and/or coating.

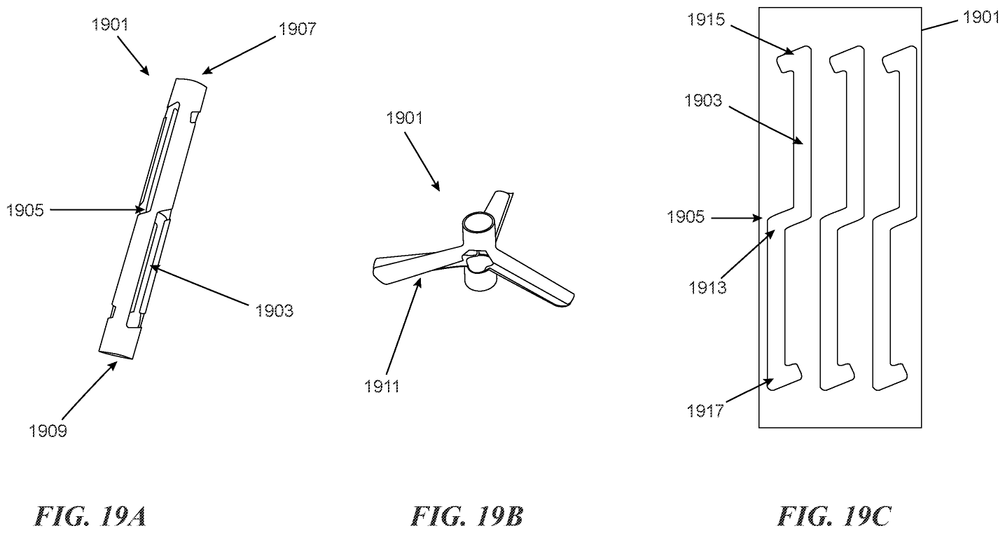

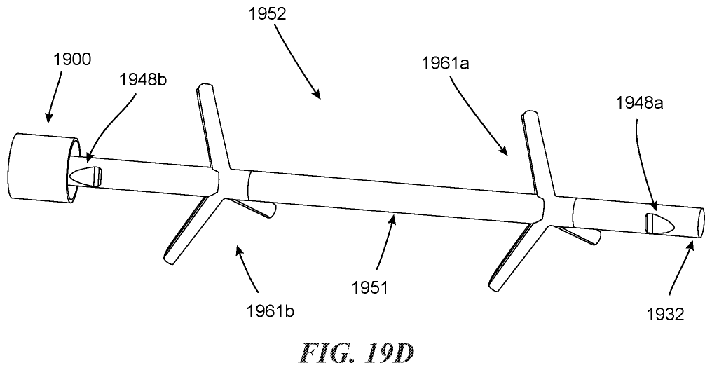

FIGS. 19A-19C illustrate an example of an expandable tube 1901 that may be part of an anchoring device. FIG. 19A shows the tube 1901 in a radially compact state, and FIG. 19B shows the tube 1901 in a radially expanded state. The tube 1901 includes a pattern of cutouts 1903 (e.g., laser cut) that are arranged such that the tube 1901 can axially collapse and form radially extending arms 1911. For example, a force applied in an axial direction from a first end 1907 toward a second end 1909 of the tube 1901 (and/or from the second end 1909 toward the first end 1907 of the tube 1901) causes a bendable region 1905 of the tube 1901 to preferentially bend outward. In this case, the bendable region 1905 is in a middle portion of the tube 1901. The cutouts 1903 are shaped such that the bendable region 1905 forms arms 1911 when expanded. In some examples, the tube 1901 is made of a shape-memory material and set to the expanded state (FIG. 19B) such that the tube 1901 takes on the expanded state when release from a catheter (e.g., delivery catheter). The diameter of the tube 1901 in the compact state and the expanded state may vary. In some examples, a ratio of the diameter of the tube 1901 in the expanded state (e.g., with arms 1911 fully extended) versus in the compact state has a value ranging anywhere from 2:1 to 15:1 (e.g., 2:1, 3:1, 4:1, 5:1, 6:1, 7:1, 8:1, 9:1, 10:1, 11:1, 12:1, 13:1, 14:1, or 15:1).

FIG. 19C shows a flattened version of the tube 1901 to show the pattern of cutouts 1903. As shown, the tube 1901 includes three elongate cutouts 1903 that run along the longitudinal axis of the tube 1901. Each of the cutouts 1903 incudes a bend 1913 in the bendable region 1905, where adjacent bends 1913 of the cutouts 1903 are closer to each other portions of the cutouts 1903. A first end of each cutout 1903 includes a first curved portion 1915, and a second of each cutout 1903 includes a second curved portion 1917. In this example, the first curved portion 1915 curves in the opposite direction as the second curved portion 1917. The shape of the cutouts 1903 may be characterized as having an “s” shape. The tube 1901 may be made of any of a number of materials. In some cases, the tube 1901 is made of a metal material. In some examples, the tube 1901 is made of a shape-memory material (e.g., nitinol).

When expanded, the arms 1911 may be used to anchor the tube 1901 (and the anchor assembly) within the ventricle wall. In some examples, the arms 1911 may be used as a one of two retaining features to retain the anchor assembly coupled to the ventricle wall. FIG. 19D shows an example an anchor 1952 that includes a tube 1951 with two sets of arms 1961a and 1961b that may act as expandable retaining features. Similar to the anchor 1202 of FIGS. 12A-12C and the anchor 1252 of FIGS. 12D-12F, the anchor 1952 includes a first expandable portion (first set of arms 1961a) and a second expandable portion (second set of arms 1961b) that are configured to expand when released from an outer sheath 1900. Although not shown in this example, in some cases, a distal tip 1932 of the tube 1951 may be sharp for penetrating tissue (e.g., through the ventricle wall). In some examples, the sets of arms 1961a and 1961b are expanded by applying an axial force on the tube 1951 (e.g., by pulling or pushing an inner wire within the tube 1951). In some examples, the sets of arms 1961a and 1961b are self-expanding when released from the sheath 1900. For example, the sets of arms 1961a and 1961b may be made of a shape-memory material (e.g., nitinol) that is heat-set to have the expanded configuration. In some examples, the sets of arms 1961a and 1961b are configured to contract into the radially compressed configuration by retraction into a sheath (e.g., outer sheath 1900). In some cases, the anchor 1952 includes one or more locking features to lock the anchor 1952 to the ventricle wall. For example, the tube 1952 may include one or more tabs (e.g., laser cut tabs) that are configured to lock within corresponding cutouts of a wire within the tube 1952. In this example, the anchor 1952 includes two locking features: a distal locking feature 1948a on a distal side of the first (e.g., distal) sets of arms 1961a, and a proximal locking feature 1948b on a proximal side of the second (e.g., proximal) sets of arms 1961b.

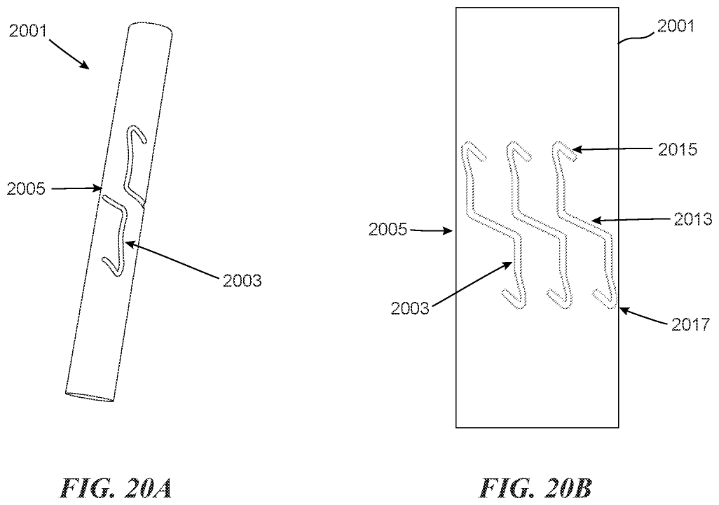

FIGS. 20A and 20B illustrate another expandable tube 2001 that is similar to the expandable tube 1901 of FIGS. 19A-19C except that the expandable tube 2001 has a different cutout 2003 pattern. Each cutout 2003 has an “s” shape, which includes a bend 2013 in a middle/bendable region 2005 of the tube 2001, a first curved portion 2015, and a second curved portion 2017. The “s” shaped cutouts 2003 in this example have shorter longitudinal lengths compared to the “s” shaped cutouts 1903 of FIGS. 19A-19C. In some examples, the expandable tube 2001 includes two expandable regions 2105 that are configured to preferentially bend and form two sets of radially expanding arms, similar to FIG. 19D.

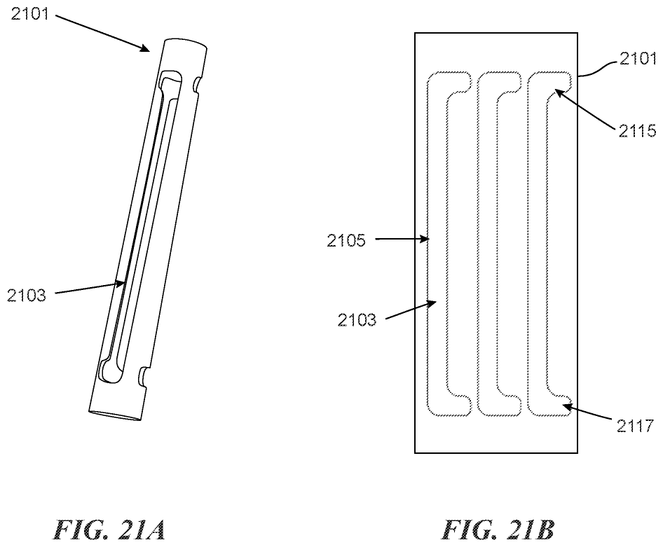

FIGS. 21A and 21B illustrate another expandable tube 2101 that is similar to the expandable tube 1901 of FIGS. 19A-19C and the expandable tube 2001 of FIGS. 20A-20C, except that the expandable tube 2101 has a cutout 2103 pattern that forms a “c” shape. Each cutout 2103 includes a first curved portion 2115 at a first end of the cutout 2103, and a second curved portion 2117 at a second end of the cutout 2103. In some examples, the expandable tube 2101 includes two expandable regions 2105 that are configured to preferentially bend and form two sets of radially expanding arms, similar to FIG. 19D.

1. A system for treating a diseased heart, the system comprising:
an annular support member that is shaped and sized for placement near a native valve annulus;
an anchor member comprising at least one anchor portion configured for implantation in tissue within a ventricle; and
at least one tether that is configured for extension from the at least one anchor portion to the support member, the at least one tether configured to apply a tensioning force between the support member and the at least one anchor portion.

2. The system of claim 1, wherein the at least one anchor portion is configured for implantation in a ventricle wall or a papillary muscle.

3. The system of claim 1, wherein the support member is configured to alter a dimension of the native valve annulus.

4. The system of claim 1, wherein the at least one tether is configured to secure the support member to the native valve annulus.

5. The system of claim 1, wherein the at least one anchor portion comprises a coil near a distal end thereof.

6. The system of claim 1, wherein the at least one anchor portion comprises a taper that reduces to a point at a distal end thereof.

7. The system of claim 1, wherein the at least one anchor portion includes an elongate tube having a first circumference, and wherein the at least one anchor portion comprises an expandable region configured to expand to a second circumference that is larger than the first circumference, wherein the expandable region is near a distal end of the at least one anchor portion.

8. The system of claim 7, wherein the at least one anchor portion comprises at least two expandable regions configured to expand to circumferences larger than the first circumference.

9. The system of claim 1, wherein the support member comprises a self-expanding material.

10. The system of claim 1, further comprising a prosthetic valve that is sized and shaped to be placed within the support member.

11. The system of claim 10, wherein the prosthetic valve is configured to transition from an unexpanded configuration to an expanded configuration.

12. The system of claim 10 wherein, upon placement within the support member, the prosthetic valve is configured to displace native valve leaflets.

13. The system of claim 1, further comprising first and second flow meters that are coupled with the support member, and that are positioned and configured for inductive coupling.

14. The system of claim 13, wherein the first and second flow meters are disposed about a periphery of the support member.

15. The system of claim 1, wherein the annular support member is expandable and includes wires arranged to form an annular shape.

16. The system of claim 15, wherein the wires define an inner wall and outer wall.

17. The system of claim 1, wherein the annular support member is conically shaped.

18. The system of claim 1, wherein the annular support member is half-dome shaped.

19. The system of claim 1, wherein the at least one anchor includes an expandable portion that is configured to expand from a first diameter to a second diameter.

20. The system of claim 1, wherein the at least one anchor includes a collapsible tube having a pattern of cutouts that is configured to collapse the tube and form radially extending arms.

21-120. (canceled)
